SEQUENCE LISTING (30 sequences)

SEQ ID NO: 1 - RNA, 91 nt, Solanum tuberosum
aauaacaagccuuccauuuucuauuuugauuuguagaaaacucgugugcuugggagucccugaugauuaaauaaaccaagauuuuaccaug

SEQ ID NO: 2 - RNA, 82 nt, Lactuca sativa
aauaacaagccuuuaguuuuccauuugaaaauucgugcgcuugggagucccugauaauuaaauaaaccaagauuuuaccaug

SEQ ID NO: 3 - RNA, 90 nt, Zea mays
aauaacaagccuucuauuaucuauuuucuuucuaguuaauacgugugcuugggaguccuugcaauuugaauaaaccaagaucuuaccaug

SEQ ID NO: 4 - RNA, 91 nt, Nicotiana tabacum
aauaacaagccuuccauuuucuauuuugauuuguagaaaacuagugugcuugggagucccugaugauuaaauaaaccaagauuuuaccaug

SEQ ID NO: 5 - RNA, 91 nt, Solanum lycopersicum
aauaacaagccuuccauuuucuauuuuuauuuguagaaaacucgugugcuugggagucccugaugauuaaauaaaccaagauuuuaccaug

SEQ ID NO: 6 - RNA, 83 nt, Helianthus annuus
aauaacaagccuuugguuuuccauuugaaaauucgugcgcuugggagucccugauaauuaaauaaaaccaagauuuuaccaug

SEQ ID NO: 7 - RNA, 82 nt, Guizotia abyssinica
aauaacaagccuuugguuuuccauuugaaaauucgugcgcuugggagucccugauaauuaaauaaaccaagauuuuaccaug

SEQ ID NO: 8 - RNA, 83 nt, Oryza sativa
aauaacaagccuucuauuaucuuucuaguuaauacgugugcuugggaguccuugcaauuugaauaaaccaagaucuuaccaug

SEQ ID NO: 9 - RNA, 86 nt, Hardeum vulgare
aauaacaagccuucuauuaucuauauucuaguuaauacgugugcuugggaguccuugcaauuugaauaaaccaagaucuuaccaug

SEQ ID NO: 10 - DNA, 171 bp, Brassica rapa
atctaaactaaaaggatcttagccattttacattggttgacatggctatataagtcatgttatactgttcaataacaagctctcaattatctacttatagttttagagaatttgtgtgcttgggagtccctgatgattaaataaaccaaggattttaccatgactgcaatt

SEQ ID NO: 11 - DNA, 178 bp, Citrus sinensis
ctgaatcggaatctagacaagatcatgaatatttatctatggattgttggttgacacggagatataagtcatgttatactgttgaataacaagccctcaattctcgctttttgcagaatttgtatgcttgggagtccctgatgattaaataaaccaagattttaccatgactgcaatt

SEQ ID NO: 12 - DNA, 154 bp, Daucus carota
gtctagagaaatattggttgacaaggatatataggtcatgttatactgttgaataacaagccttcagttttctatatttcgagttatagaaaattcgtgtgcttgggagtccctgataattaaataaaccaagatttttaccatgactgctatt

SEQ ID NO: 13 - DNA, 178 bp, Gossypium hirsutum
atctagatacgatcctagatgtatattttctattttctatttgatattggttgacacgggtatataagtcatgttatactgttgaataacaagccttcaattatctagttctagataatttgtgcgcttgggagtccctgatgattaaataaaccaagattttaccatgactgcaatt

SEQ ID NO: 14 - DNA, 142 bp, Helianthus annuus
acccagctagtgttggttgacacgcgcatataagtcatgttatactgttaaataacaagcctttggttttccatttgaaaattcgtgcgcttgggagtccctgataattaaataaaaccaagattttaccatgactgcaatt

SEQ ID NO: 15 - DNA, 151 bp, Solanum lycopersicum
atccacatataccttggttgacacgagtatataagtcatgttatactgttgaataacaagccttccattttctatttttatttgtagaaaactcgtgtgcttgggagtccctgatgattaaataaaccaagattttaccatgactgcaatt

SEQ ID NO: 16 - DNA, 176 bp, Vitis vinifera
atctagatagataatagatatatctgtggatgttgatattggttgacacaggcatataagtcatgttatactgttgaataacaagccttcaattatctatttctatttttagataattcgtgtgcttgggagtccctgatgattaaataaaccaagattttaccatgactgtaatt

SEQ ID NO: 17 - DNA, 183 bp, Manihot esculenta
atctagatagataagctcataaagatatctctatgaatgttggtgttggttgacacgggtatataagtcatgttatactgttgaataacaagccctcaattatctatttcgatttatagagaattcgtgtgtttgggagtccctgatgattaaataaaccaagattttaccatgactgcaatt

SEQ ID NO: 18 - DNA, 173 bp, Sorghum bicolor
atgaagttatacgaaaatccaattgttggggctggcttggttgacattggtatatagactatgttatactgttaaataacaagccttctattatctattttctttctagttaatacgtgtgcttgggagtccttgcaatttgaataaaccaagatcttaccatgactgcaatt

SEQ ID NO: 19 - DNA, 173 bp, Zea mays
atgaagttatacgaaaatccaatcgttggggctggcttggttgacattggtatatagactatgttatactgttaaataacaagccttctattatctattttctttctagttaatacgtgtgcttgggagtccttgcaatttgaataaaccaagatcttaccatgactgcaatt

SEQ ID NO: 20 - DNA, 161 bp, Hardeum vulgare
tatatggatagccaatctttgggctgacttggttgacattggtatatagtctatgttatactgttaaataacaagccttctattatctatattctagttaatacgtgtgcttgggagtccttgcaatttgaataaaccaagatcttaccatgactgcaatt

SEQ ID NO: 21 - DNA, 169 bp, Glycine max
atctaaataagagcatacttactatggatattggtattggttgacactggtatataagtcatgttatactgttgaataacaagtcctcaaattttctaattctagataattttggtgcttgggagtccctaatgattaaatgaaccaagattttaccatgactgcaatt

SEQ ID NO: 22 - DNA, 142 bp, Lactuca sativa
atccagatagatattggttgacacgggcatataagtcatgttatactgttaaataacaagcctttagttttccatttgaaaattcgtgcgcttgggagtccctgataattaaataaaccaagattttaccatgactgcaatt

SEQ ID NO: 23 - DNA, 151 bp, Nicotiana tabacum
atctacatacaccttggttgacacgagtatataagtcatgttatactgttgaataacaagccttccattttctattttgatttgtagaaaactagtgtgcttgggagtccctgatgattaaataaaccaagattttaccatgactgcaatt

SEQ ID NO: 24 - DNA, 148 bp, Coffea Arabica
atctagatcgatattggttgacacaagtatataaggcgtgttatactgttgaataacaagccttccactttctatttgaatttgtgtgcttgggagtccctgatgattaaataattaaataaaccaagattttaccatgactgcaatt

SEQ ID NO: 25 - DNA, 175 bp, Cucumis sativus
atctagataactaataaaagcatatggatgttgatattggttgacatgggtatataagtcatgttatactgttgaataacaagcccttcattttctatttctagagaattcatgtgcttgggagtccctgatgattaattattaaataaaccaagattttaccatgactgcaatt

SEQ ID NO: 26 - DNA, 149 bp, Spinacia oleracea
atctcactagatattggttgacacgggcatataaggcatgttatactgttgaataacaatctttcaatttctatttctagcgaatttgtgtgcttgggagtccctgatgattaaattaaataaaccaagattttaccatgactgcaatt

SEQ ID NO: 27 - DNA, 174 bp, Cicer arietinum
atctagatgaataacagcatacaatggatattggtattggttgacaccagtatataagtcatgttatactgttgaataacaagccctttattatctagttatagataattcgtgtgcttgggagtccctgataattaaatttaaataaaccgagattttaccatgactgcaatt

SEQ ID NO: 28 - DNA, 173 bp, Medicago truncatula
aatctagatcaaaaacaggacaatggatattgatattggttgacaccggtatataagtcatgttatactgttgaataacaagcccttaattctatacttatagagaattcgtgtgcttgggagtccctgataattaaatttaaataaaccaagattttaccatgactgcaatt

SEQ ID NO: 29 - DNA, 163 bp, Oryza sativa
ttgttgtatggatatacaactgtcggggctggcttggttgacattggtatatagtctatattatactgttaaataacaagccttctattatctttctagttaatacgtgtgcttgggagtccttgcaatttgaataaaccaagatcttaccatgactgcaatt

SEQ ID NO: 30 - DNA, 24 bp, Artificial Sequence
  Description of Artificial Sequence Synthetic primer
cagcagccgcggtaatacagagga

(19) United States
(12) Patent Application Publication
(10) Pub. No.: US 2014/0101792 A1
(43) Pub. Date: Apr. 10, 2014

(54) PLASTID TRANFORMATION UTILIZING ENDOGENOUS REGULATORY ELEMENTS

(71) Applicant: Henry Daniell, Winter Park, FL (US)

(72) Inventor: Henry Daniell, Winter Park, FL (US)

(73) Assignee: University of Central Florida Research Foundation, Inc., Orlando, FL (US)

(21) Appl. No.: 13/865,146

(22) Filed: Apr. 17, 2013

Related U.S. Application Data
(63) Continuation of application No. 12/795,572, filed on Jun. 7, 2010, now abandoned.
(60) Provisional application No. 61/184,673, filed on Jun. 5, 2009.

(51) Int. Cl. C12N 15/82

(52) U.S. Cl. CPC C12N 15/8261
USPC 800/298; 435/320.1

(57) ABSTRACT

Disclosed herein are vectors, plants and methods of transforming plants to increase translation efficiency. Specifically disclosed are methods of implementing a regulatory element endogenous to a target plant species and operatively associating said regulatory element with a heterologous gene of interest. Examples of regulatory sequences are disclosed including 5′ UTR sequences of chloroplast genes, such as a psbA gene.

### CROSS-REFERENCE TO RELATED APPLICATIONS

This application is a continuation of U.S. Ser. No. 12/795,572 filed Jun. 7, 2010 which is related to U.S. Provisional Application No. 61/184,673 filed Jun. 5, 2009 and to which priority is claimed under 35 USC 119 and the entirety of the noted application is incorporated herein.

### INTRODUCTION

Over the expanse of time since the endosymbiotic events that led to the establishment of plant organelles, plant cells have evolved elaborate mechanisms to coordinate the expression of plastid genes with the changing developmental and functional requirements of the cell. In addition to the nuclear encoded, plastid localized RNA polymerase (NEP), the nucleus controls the expression of a suite of σ-factors required for the active transcription of photosynthetic genes by the plastid encoded RNA polymerase (PEP), with PEP itself being transcribed by NEP (Allison et al., 1996; Hess and Borner, 1999). Nuclear control over translation of plastid mRNA is exerted through the activities of numerous plastid-localized RNA binding proteins (RBPs). RBPs appear to have a tight affinity for their cognate sequences in plastid mRNAs and studies have demonstrated that their interactions are specific for particular genes (Nakamura et al., 1999, 2001; Shen et al., 2001; Meierhoff et al., 2003; Schmitz-Linneweber et al., 2005, 2006).

Plastid mRNAs, expressed as mono- or polycistrons, contain 5′ and 3′ untranslated regions (UTRs). Detailed analyses have demonstrated that within these UTRs lie cis elements, often forming secondary structures, which facilitate the interaction with nuclear encoded RBPs (Yang et al., 1995; Hirose and Sugiura, 1996; Klaff et al., 1997; Alexander et al., 1998; Zou et al., 2003; Merhige et al., 2005;). RBPs display an array of functions including processing of polycistronic transcription units, RNA maturation and editing, transcript stability and turnover and the recruitment of additional protein factors involved in initiation of translation in response to the demands of the cell (Nickelsen, 2003; Schimtz-Linneweber and Barkan 2007). In contrast to the high level of conservation found within protein coding regions and ribosomal RNAs, intergenic and untranslated regions are highly variable in chloroplast genomes (Daniell et al., 2006; Saski et al., 2007; Timme et al., 2007)

Chloroplast transformation strategies have utilized both endogenous and foreign regulatory elements to facilitate high levels of foreign gene expression. Hybrid systems comprising a modified tobacco (Nicotiana tabacum) ribosomal operon promoter (Prrn) in conjunction with a translational control region (TCR) derived from the N. tabacum plastid-encoded rbcL gene or from bacteriophage T7 gene 10 (g10) to express foreign genes have been utilized in numerous species (Guda et al., 2000; Kuroda and Maliga, 2001; Ruhlman et al., 2007). Another approach incorporates the native psbA 5′ and 3′ UTRs into transformation constructs (Verma et al., 2008). The potential of psbA 5′ UTR stems from its important role in plastids. Photosystem II core protein D1 is a polytopic thylakoid membrane constituent with five membrane-spanning helices encoded by the plastid psbA gene (Marder et al., 1987). Expression of D1 is predominantly regulated at the level of translation and requires the participation of RBPs imported into plastids post-translationally from the cytoplasm. Photosystem II is highly susceptible to excessive light and the primary target of the damage is D1. If the core protein is not efficiently removed and replaced the result is impairment of electron transport, known as photoinhibition (Yamamoto, 2001). It is this cycle of turn over that makes the psbA 5′ UTR an attractive tool to enhance the level of foreign gene expression in transplastomic lines. The use of endogenous psbA regulatory elements has facilitated the generation of transplastomic N. tabacum lines with enhanced expression of a large number of soluble (Verma et al., 2008) and membrane proteins (Singh et al., 2009), generating transplastomic lines conferring desired agronomic traits (Bock, R., 2007; Daniell et al., 2005; Verma and Daniell, 2007) or expressing biopharmaceutical proteins and vaccine antigens (Davoodi-Semiromi et al., 2009).

However, overwhelming majority of foreign proteins has been expressed in N. tabacum chloroplasts. In order to advance this field, chloroplast genomes of several crop species should be transformed. In addition, rapid and reproducible oral delivery systems expressing vaccine antigens, autoantigens or biopharmaceuticals should be developed. There are two major limitations in accomplishing these goals.

Development of direct organogenesis system is important for establishment of efficient and reproducible plastid transformation systems in crops regenerated via organogenesis. In addition, improvement in our understanding of the role of endogenous or heterologous regulatory sequences in transgene expression in plastids is essential.

### BRIEF DESCRIPTION OF THE DRAWINGS

FIG. 1: Nucleotide alignment of the psbA 5′ UTR. IGS were extracted from complete plastid genomes of 20 species of angiosperm. Colored bases indicate agreements. Promoter & transcription start, purple; stem loop region, grey; RBS, yellow; translational start, blue. Complete data from genomic analyses are available at (http://chloroplast.cbio.psu.edu/supplement.html).

FIG. 2: Schematic representation of expression cassettes and confirmation of homoplasmy by Southern hybridization: A) Schematic representation of the chloroplast transformation vectors. Ls, L. sativa, Nt, N. tabacum, Prrn, rRNA operon promoter; aadA, aminoglycoside 3′-adenylytransferase gene; TrbcL, 3′ untranslated region of rbcL, Trps16, 3′ untranslated region of rps16, g10, 5′ translation control element of bacteriophage T7 gene10, CTB-Pins, coding sequence of CTB subunit fused to human proinsulin, PAG, coding sequence of Bacillus anthracis protective antigen gene, PpsbA, promoter and 5′ untranslated region of psbA gene; TpsbA, 3′ untranslated region of psbA gene. Homoplasmic transformants generated for this study B) Ls-Nt-CP, C) Ls-Ls-PA and D) Ls-g10-PA. For B, C &D: Lane 1, wild type; (B: 4.2 kb; C&D 3.1 kb) lanes 2 & 3, independent transplastomic lines (B: 6.4 kb; C&D: 7.1 kb).

FIG. 3: Accumulation of foreign protein in transplastomic L. sativa and N. tabacum. A) CTB-Pins accumulation estimated by densitometry and B) PA accumulation estimated by ELISA presented as a function of light and developmental stage. C) SDS-PAG stained with Coomassie blue. Lanes 1,3,5) 10, 20, 30 μg TLP from Nt-Nt-CP older leaf at 6 pm; lanes 2,4,6) corresponding amount of wild type protein extract; L is molecular weight standard.

FIG. 4: Northern blotting of total RNA. To examine foreign transcript abundance in transplastomic lines 2.5 μg of total RNA was separated by electrophoresis blotted to nylon membranes and probed with radiolabeled CTB-Pins or PA fragment. A)

Upper frame, autoradiographs; lower frame; ethidium bromide stained rRNA. Lanes 1) Ls-g10-CP; 2) Nt-Nt-CP; 3) Ls-Nt-CP; 4) Nt-Nt-PA; 5) Nt-Ls-PA; 6) Ls-Ls-PA; 7) Ls-g10-PA. B) Desitometric quantitification for abundance of foreign transcripts in lines with heterologous regulatory elements relative to those with native elements.

FIG. 5: Polysome assay. A) Sucrose gradient fractions were separated through 1.2% agarose and transferred for northern blotting with CTP-Pins probe. Lanes are numbered above A) for both A (Ls-Ls-CP) and B (Ls-Nt-CP). Lane 1) total RNA, 2) blank, 3-14) fractions 1-12 collected from the bottom of the gradient, 15) RNA standards 16) CTB-Pins probe. C) Lanes 1-4 and 5-8) fractions 1, 4, 8, 11 from Ls-Nt-CP and Ls-Ls-CP, respectively. D) Controls; lanes 1-3) pooled fractions from wt sample, 4) blank. Lanes 5-7) each lane contains 2 pooled factions from 2-7 of puromycin-treated sample (Ls-Ls-CP). In lanes 8-12 fraction corresponds to lane number

FIG. 6: RNA EMSA-Competition assay and mRNA turnover. Stromal proteins isolated from L. sativa (A) or N. tabacum (B) were incubated with radiolabeled native psbA 5′ UTR (lane 1). Competitions included 50× unlabeled native psbA 5′ UTR (lane 2) and 50, 100 or 200× unlabeled foreign psbA UTR (N. tabacum in A, or L. sativa in B; lanes 3-5, respectively). Lane 6 shows labeled probe. Brackets indicate free probe, arrows indicate complexes associated with labeled RNA. C) ³²P LsUTR-CTB-Pins mRNA was incubated in either Ls or Nt stromal extracts for the time indicated on X axis and extracted RNA was separated by electrophoresis; upper panel shows autoradiographs of the ˜800 base UTR-CTB-Pins transcript in each background and (lower) shows graphic representation of two independent experiments. Error bars are standard deviation for two experiments.

FIG. 7: Stability of CTB-Pins in transplastomic plants. Total translation products were labeled for 1 hour with ³⁵S and samples were taken over a time course as shown on the X axis. Protein extracts were immunoprecipitated with α-Cholera toxin antibody and analyzed by scintillation counting.

FIG. 8. Theoretical predictions of secondary structure within the psbA 5′ UTR. The Vienna RNA Websuite tool, RNAfold, was used to produce two-dimensional structures for the psbA 5′ UTR for three representatives each from Solanaceae (top row), Asteraceae (middle row), and Poaceae (bottom row), based on minimum free energy. Unpaired bases upstream and downstream of the predicted stem and loop region are shown as circular, with 5′ and 3′ ends labeled accordingly; this representation is arbitrary and software dependent and is not intended as an indication of structure. AG values are given below the species names for each structure. Arrowheads indicate potential ribosome-binding sites (RBS). Small arrows indicate base changes in RBS relative to tobacco; these sites are identical within the families shown. Brackets indicate the AU box.

FIG. 9. Schematic representation of expression cassettes and confirmation of homoplasmy by Southern hybridization. A, Schematic representation of the chloroplast transformation vectors. Ls, L. sativa; Nt, N. tabacum; Prrn, rRNA operon promoter; aadA, aminoglycoside 3′-adenylytransferase gene; TrbcL, 3′ UTR of rbcL; Trps16, 3′ UTR of rps16; g10, 5′ translation control element of bacteriophage T7 gene 10; CTB-Pins, coding sequence of CTB subunit fused to human proinsulin; pagA, coding sequence of Bacillus anthracis protective antigen gene; PpsbA, promoter and 5′ UTR of psbA gene; TpsbA, 3′ UTR of psbA gene. B to D, Homoplasmic transformants generated for this study: Ls-Nt-CP (B), Ls-Ls-PA (C), Ls-g10-PA (D). Lane 1, The wild type (B, 4.2 kb; C and D, 3.0 kb); lanes 2 and 3, independent transplastomic lines (B, 6.4 kb; C and D, 7.1 kb). Five micrograms of total DNA was digested completely with Af/III for the Ls-Nt-CP blot and with SmaI for the Ls-Ls-PA and Ls-g10-PA blots.

### DETAILED DESCRIPTION

According to one embodiment, the invention pertains to a stable plastid transformation and expression vector which comprises an expression cassette optimized for expression in a target plant species. The expression cassette includes a regulatory sequence that is endogenous to the target plant species, a heterologous polynucleotide sequence of interest, transcription termination sequence functional in said plastid, and flanking each side of the expression cassette, flanking DNA sequences which are homologous to a DNA sequence of the target plastid genome. The vector enables the stable integration of the heterologous coding sequence into the plastid genome of the target plant that is facilitated through homologous recombination of the flanking sequence with the homologous sequences in the target plastid genome. Optionally, the expression cassette optionally includes a heterologous marker sequence.

Certain embodiments disclosed herein are based on the inventor's development of direct plastid transformation and plant regeneration system that enables more efficient and reproducible plastid transformation, as well as the inventor's research of the role of endogenous or heterologous regulatory sequences in transgene expression in plastids is essential. Nucleotide variability in the regions upstream of chloroplast genes that comprise promoters and UTRs was investigated. Coding and non-coding sequences across 20 crop species representing most major clades of angiosperms including 4 grasses and 3 legumes were studied. Furthermore, L. sativa and N. tabacum transplastomic lines regulating transgenes with endogenous or heterologous regulatory elements were used to investigate RNA-protein interaction, foreign transcript accumulation and polyribosome association (polysome assay), and foreign protein accumulation. The inventor has discovered that species-specific optimization of plastid transformation vectors and optimization of growth hormone requirement has a significant impact on foreign gene expression and transformation efficiency.

In a specific embodiment, the invention pertains to a chloroplast expression vector that implements a regulatory sequence that is endogenous to the target plant species, where the regulatory sequence relates to a 5′ UTR sequence of a chloroplast gene. Unless specified otherwise, the term 5′ UTR is intended to pertain to a native 5′ UTR and is construed to pertain to variants or fragments thereof. In an even more specific embodiment, the regulatory sequence relates to a 5′ UTR sequence of the psbA chloroplast gene, or fragment or variant thereof.

Provided herein are accession numbers for sequenced plastid genomes of several different species, see Table 3. Also, Attachment A includes a listing of psbA genes for different target species and their location in the plastid genome, as well as the accession no. for the genome. The location of the psbA gene in the genome is noted in Attachment A for each entry. Moreover, using Arabidopsis thaliana as a specific example, as shown on page 1 of Attachment A, it shows the genome accession no. NC_{—}000932.1 and the location of the gene at by 383..1444. Unless specified otherwise, the 5′ UTR sequence comprises 200 base pairs (bp) or less upstream of the translation initiation codon. This guideline of identifying the 5′ UTR sequence for the psbA genes can be applied to other chloroplast genes. Table 2 lists common genes encoded by chloroplast genomes. Thus, broadly speaking, the 5′ UTR of these noted genes may be used as regulatory sequences.

As noted above, when considering the psbA gene or other chloroplast genes, the 5′ UTR comprises sequences 200 by or less upstream of the translation initiation codon. In more specific embodiments, the 5′ UTR is 10-100 by upstream of the initiation codon. In an even more specific embodiment, the 5′UTR is 10-70 by upstream of the initiation codon.

According to certain embodiments, the target plant species include, but are not limited to, cereals such as barley, corn, oat, rice, and wheat, melons such as cucumber, muskmelon, and watermelon; legumes such as bean, cowpea, pea, peanut; oil crops such as canola and soybean; solanaceous plants such as tobacco, tuber crops such as potato and sweet potato, and vegetables like tomato, pepper and radish; fruits such as pear, grape, peach, plum, banana, apple, and strawberry; fiber crops like the Gossypium genus such as cotton, flax and hemp; and other plants such as beet, cotton, coffee, radish, commercial flowering plants, such as carnation and roses; grasses, such as sugar cane or turfgrass; evergreen trees such as fir, spruce, and pine, and deciduous trees, such as maple and oak. Of greatest present interest are the major economically important crops like maize, rice, soybean, wheat and cotton, and also including Lactuca sativa, or any of the crops provided on the attached tables whose genomes have been sequenced.

A “fragment” of a polynucleotide sequence provided herein is a subsequence of contiguous nucleotides that is capable of specific hybridization to a target of interest, e.g., a sequence that is at least 15 nucleotides in length. The fragments of the invention comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods of the invention.

The polynucleotide fragments of the invention may be produced by techniques well-known in the art such as restriction endonuclease digestion and oligonucleotide synthesis.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 20 nucleotide positions, preferably at least 50 nucleotide positions, more preferably at least 100 nucleotide positions, and most preferably over the entire length of the specified polynucleotide sequence. In the context of UTR sequences, the comparison window will naturally be upstream of the initiation codon of a given chloroplast gene.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:
bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/).

The similarity of polynucleotide sequences may be examined using the following unix command line parameters:
bl2seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰ , more preferably less than 1×10⁻⁶⁰, more preferably less than 1×10⁻⁷⁰, more preferably less than 1×10⁻⁸⁰, more preferably less than 1×10⁻⁹⁰ , more preferably less than 1×10⁻¹⁰⁰, more preferably less than 1×10⁻¹¹⁰, and most preferably less than 1×10⁻¹²⁰ when compared with any one of the specifically identified sequences.

Variants and homologs of the nucleic acid sequences described above also are useful nucleic acid sequences. Alternatively, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.-16.6(log₁₀ [Na⁺ ])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Relevant articles on genetic sequences is provided: proinsulin (Brousseau et al., Gene, 1982 March;17(3):279-89; Narrang et al, Can J Biochem Cell Biol. 1984 April;62(4):209-16; and Georges et al, Gene 27 (2), 201-211 (1984); and CTB (Shi et al, Sheng Wu Hua Hsueh Tsa Chih 9 (No.4), 395-399 (1993).

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis in the presence of tunicamycin, etc. Further, a derivative of a proteinaceous agent may contain one or more non-classical amino acids. A derivative of a proteinaceous agent possesses a similar or identical function as the proteinaceous agent from which it was derived. The term “derivative” in the context of a proteinaceous agent also refers to a proteinaceous agent that possesses a similar or identical function as a second proteinaceous agent (i.e., the proteinaceous agent from which the derivative was derived) but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy. In a specific embodiment, a derivative is a functionally active derivative.

### EXAMPLES

### Example 1

### Cultivars of L. sativa Respond Differently to PGRs and Regeneration Conditions

Direct shoots emerged from the leaf explants 21 days after culture in optimized regeneration medium. The shoot regeneration response was variable with different combinations of auxin and cytokinin (Table 1). We observed that auxin or cytokinin alone did not initiate shoot regeneration. A combination of auxin (NAA) and cytokinin (BAP) was necessary to induce shoot regeneration in different cultivars. Maximal direct shoot initiation was observed from the leaf explants cultured in medium supplemented with 0.1 μg mL⁻¹ NAA and 0.2 ug ml⁻¹ BAP. Other combinations of PGRs induced callus formation and decreased shoot regeneration in all cultivars. We also observed differences in responsiveness among cultivars. Simpson Elite (LS) showed the greatest regeneration potential (average 4.3 shoots per explant), whereas Lentissima (LL) had the least response (0.43 shoots per explants). Regardless of the cultivar studied, the medium supplemented with 0.1 μg ml⁻¹ NAA and 0.2 μg ml⁻¹ BAP induced the greatest number of shoots (Table 1). Following LS in regeneration efficiency were Romaine (LR; 2.5), Great Lake (LG; 1.5), and Evola (LE; 1.2). Therefore, LS cultured on LR medium with 0.1 μg ml⁻¹ NAA and 0.2 μg ml⁻¹ BAP was chosen for all of our transformation experiments.

### Example 2

### Upstream Sequence Variation Predominates in Plastid Genes Across Taxa

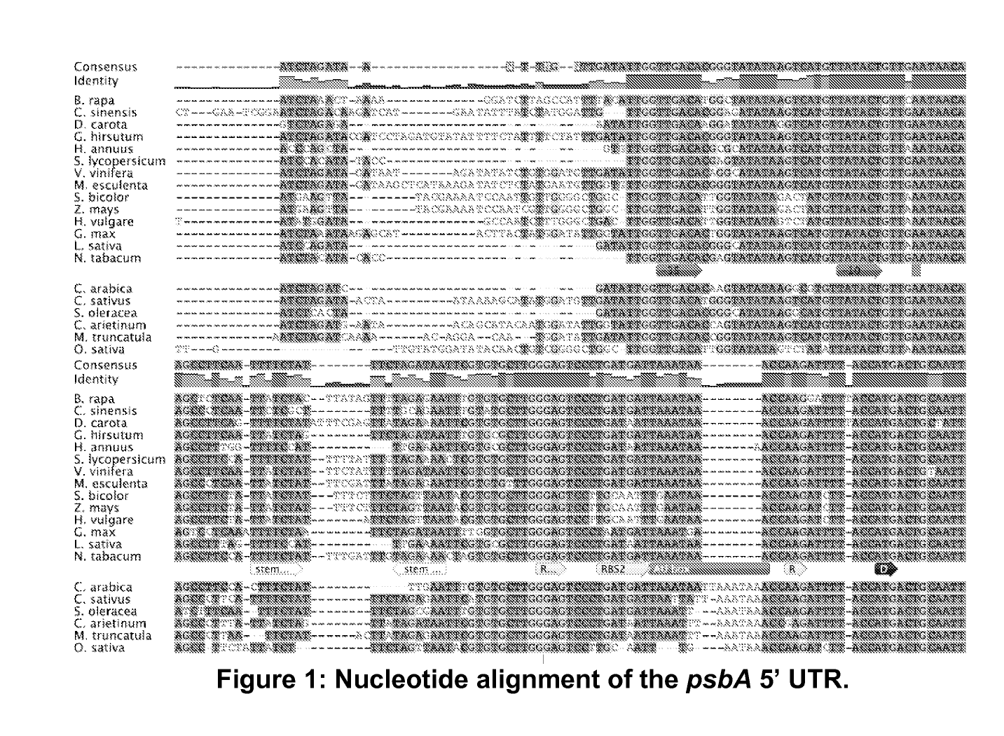

Sequences of IGS upstream from genes representing different functional groups were extracted from 20 complete plastid genomes representing most major clades of angiosperms, including 4 grasses and 3 legumes. Alignments were anchored by the inclusion of 100 bases from the coding region of adjacent genes. Sequence identity was calculated for the region encompassed by 200 bases upstream of the translation start codon, i.e. promoters and UTRs. We observed that coding regions across all genera and genes displayed sequence identity of 80% to 97%, whereas the non-coding regions were 45% to 79%. In keeping with our findings throughout this study employing the psbA 5′ UTR as an experimental model system we found that despite 95.0% identity in the coding region, identity in the psbA upstream region is 59% across all taxa (FIG. 1). The cis feature located at position -49 to -71 in relation to the N. tabacum start codon has been shown experimentally to be an important determinant of translation efficiency (Eibl et al., 1999; Zou et al., 2003). Across all taxa sequence identity for this element was 61%; N. tabacum and L. sativa have 54.2% identity for this region. All alignments have been deposited in the Chloroplast Genome Database (http://chloroplast.cbio.psu.edu/) for public access.

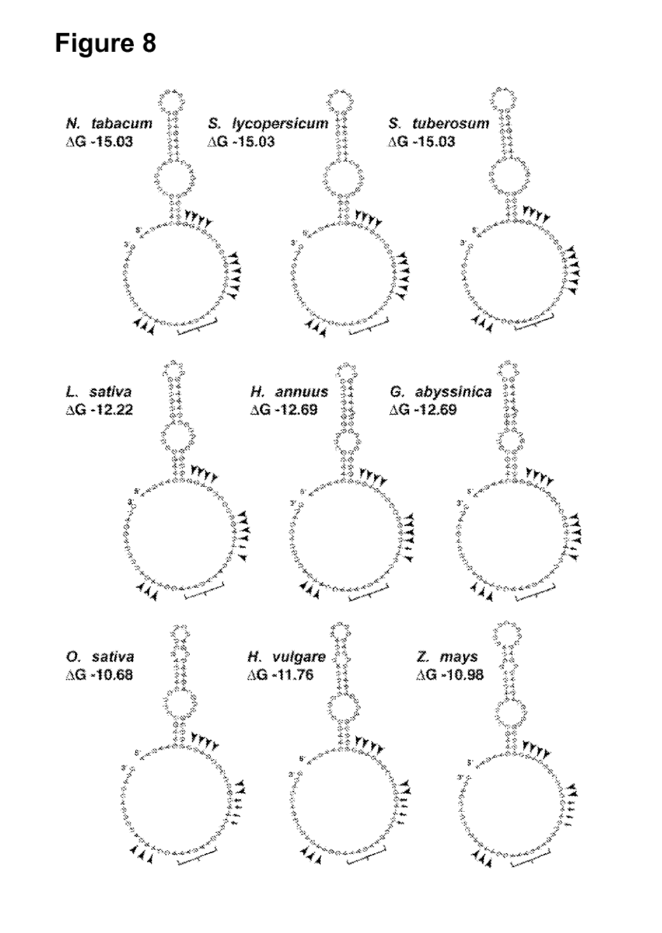

To determine how the nucleotide sequence variability in this region affects the secondary structure of the UTR, we used RNAfold to generate two-dimensional structure predictions based on minimum Gibbs free energy (ΔG; FIG. 8). Within families (Asteraceae, Solanaceae, Poaceae), stem and loop structures were highly similar, and predicted ΔG values showed little or no variation. However, among these three families, differences in nucleotide sequences result in structural and corresponding ΔG value variations. Relative to tobacco, the lettuce terminal stem and loop are reduced by one pair and three bases, respectively. Also, the base composition and conformation of the pairs within the stem are not conserved. Likewise, the stem bulge is reduced by two bases on each side, and the composition in this region is highly dissimilar. All alignments have been deposited in the Chloroplast Genome Database (http://chloroplast.cbio.psu.edu/) for public access.

### Example 3

### Plastid Transformation Vectors

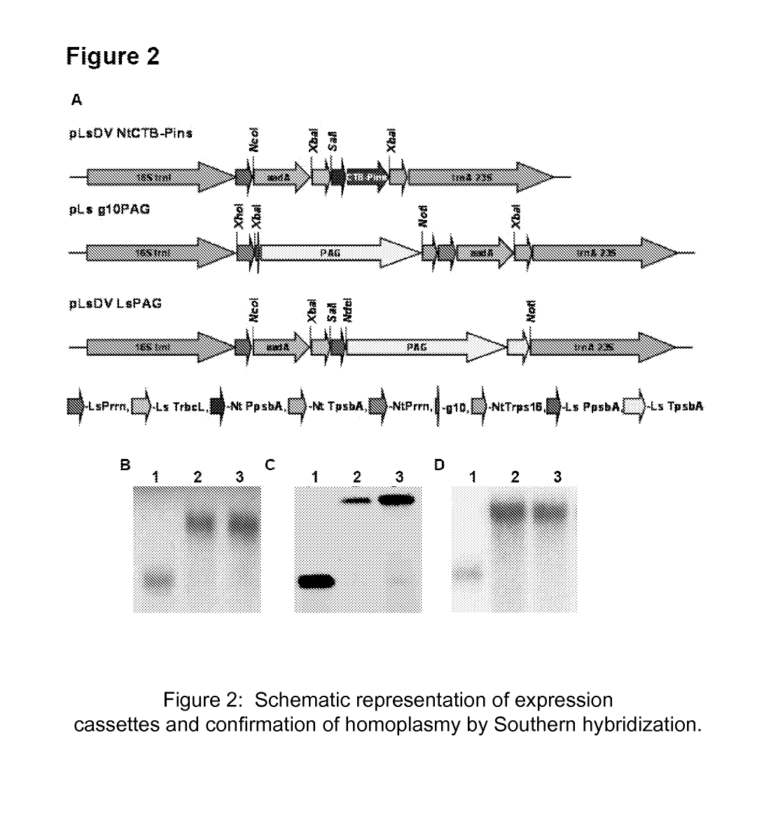

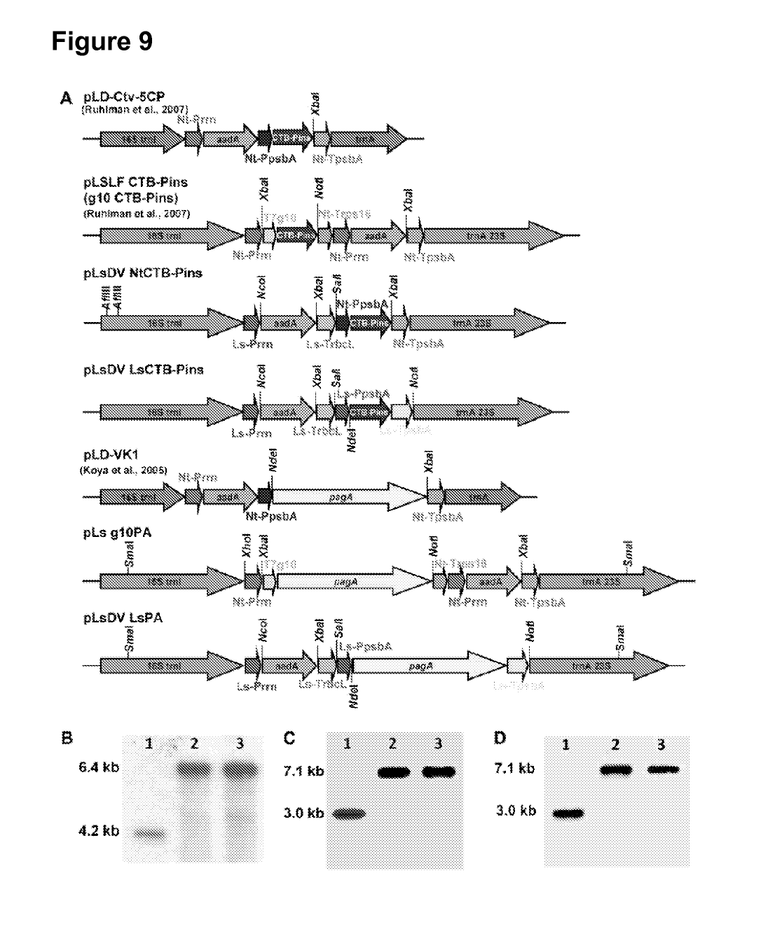

The pUC based L. sativa long flanking sequence vector was used to integrate foreign genes into the intergenic spacer region between the trnI and trnA genes as described previously (Ruhlman et al., 2007). The L. sativa native 16S ribosomal operon promoter (Prrn) and 3′ rbcL were amplified from the L. sativa plastid genome and used to regulate the expression of the aadA gene from the GGAG ribosome binding site for spectinomycin resistance. The aadA expression cassette was inserted into the spacer region between the trnl and trnA and resulted in pLsDV vector. The pLsDV NtCTB-Pins and pLsDV LsCTB-Pins plasmids were constructed by transferring the CTB-Pins expression cassette with N. tabacum and L. sativa psbA regulatory sequences to pLsDV. pLsg10 PAG was constructed by replacing CTB-Pins with pag in pLS CTB-Pins vector. The pLsDV LsPAG construct was made by cloning pag expression cassette with L. sativa psbA regulatory sequences to the pLsDV vector. The plasmids pLD VK1, pLD-5′UTR-CTB-Pins and pLS CTB-Pins used to generate Nt-Nt-PA, Nt-Nt-CP and Ls-g10-CP plants have been reported previously (FIG. 2A; Koya et al., 2005; Ruhlman et al., 2007). See also FIG. 9.

### Example 4

### Generation of Transplastomic Lines and Confirmation of Homoplasmy

Plastid transformation of L. sativa and N. tabacum was carried out as described (Daniell et al., 2005; Ruhlman et al., 2007), with modifications of regeneration media as described above. Primary regenerants identified by PCR were subjected to an additional round of regeneration followed by rooting in selective media. Site specific integration and homoplasmy of the transplastome was confirmed by Southern hybridization with flanking sequence probes specific for L. sativa or N. tabacum. Generation and confirmation of homoplasmy for CTB-Pins lines Nt-Nt-CP, Ls-g10-CP, and PA line Nt-Nt-PA have been reported previously (Koya et al., 2005; Ruhlman et al., 2007). All newly generated transplastomic lines were found to be homoplasmic, containing no detectable wild type copies of the plastome (FIGS. 2B, C & D).

### Example 5

### Decrease of Foreign Protein Accumulation with Heterologous Regulatory Elements

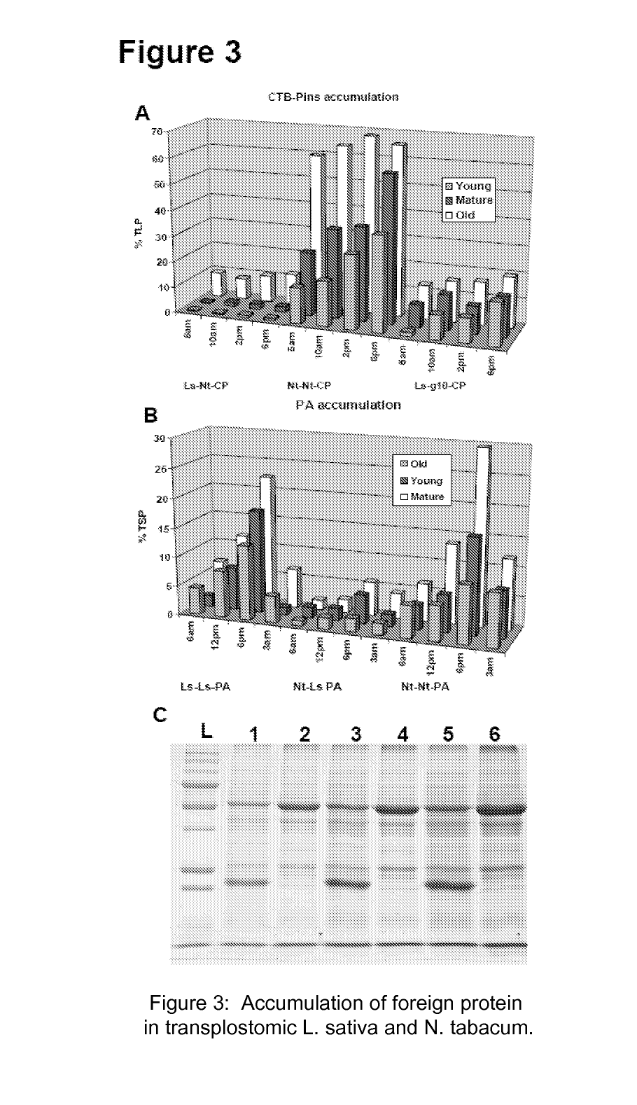

Second generation (T1) transplastomic homoplasmic plants of L. sativa and N. tabacum expressing CTB-Pins or PA were grown in the greenhouse for 8-10 weeks. Leaves of different developmental stages were harvested at four time points during the light cycle to evaluate foreign protein accumulation. Expression of CTB-Pins was quantified by densitometric analysis of crude homogenates against known quantities of CTB standard (FIG. 3A). For these quantitative studies homogenates have been used as we found that up to 90% of CTB-Pins protein is retained in the pelleted fraction following centrifugation. We modified our protein extraction and SDS sample loading buffer to include 100 mM DTT, and increased the buffer to tissue ratio to enhance solubility of CTB-Pins. In all three lines analyzed, the older leaves showed the highest levels of CTB-Pins accumulation, up to 72% of total leaf protein (TLP) in the N. tabacum line Nt-Nt-CP, 12.3% of TLP in Ls-Nt-CP and ˜25% of TLP in Ls-g10-CP. To confirm this estimate for N. tabacum we quantified samples by densitometric analysis of Coomassie stained gels. We estimated that in these lines CTB-Pins accumulation exceeds that of Rubisco by a factor of up to two (FIG. 3C) yet no deleterious phenotype was observed, likely as this level of accumulation was attained over time (Bally et al., 2009). In mature leaves harvested at 6 pm the L. sativa line accumulated CTB-Pins to 2.2% +/−0.8 of TLP whereas the N. tabacum line reached 57% +/−2.1, a 96% reduction in foreign protein, despite the fact that both of these lines express CTB-Pins from the N. tabacum psbA 5′ UTR. The expression pattern observed in Nt-Nt-CP young and mature leaves was consistent with developmental and light regulation with increasing levels of foreign protein as maturing leaves synthesize and accumulate proteins throughout the light cycle. Minimal differences were observed in the two L. sativa lines and were attributed to the overall physiological condition of the plants and experimental variation.

Expression of PA in transplastomic N. tabacum and L. sativa was determined by ELISA of young, mature and older leaves. PA was mostly observed in the soluble fraction and therefore quantitation by ELISA was accurate. The maximum PA expression was observed in mature leaves compared to young or older leaves in all the lines examined. In mature leaves harvested at 6 pm from L. sativa and N. tabacum lines, PA accumulation reached up to 22.4% +/−1.0 and 29.6% +/−0.9 of total soluble protein (TSP) respectively, when PA expression was regulated by endogenous regulatory elements. In Nt-Ls-PA, the foreign protein represented 5.8% +/−0.1 of TSP in mature leaves at 6 pm, an 80% reduction in expression in N. tabacum (FIG. 3B). In order to investigate whether this reduction in expression level was the result of any deletion or modification of the regulatory elements or coding sequence of the PA expression cassette, DNA from Nt-Ls-PA line was isolated and nucleotide sequence of the expression cassette was determined. Nucleotide sequence of the PA expression cassette in the bombarded chloroplast expression vector and the Nt-Ls-PA transplastomic chloroplast genome was identical (data not shown), confirming absence of any recombination or modification of the psbA regulatory elements or the PA coding sequence. Like the CTB-Pins lines, PA accumulation followed predictable developmental and light regulated patterns when endogenous psbA regulated transgene expression. CTB-Pins and PA accumulated foreign gene products differently in older leaves. While CTB-Pins was present in the highest levels, PA expression was the lowest in older leaves probably due to protection from proteolytic degradation conferred by aggregation of CTB-Pins.

### Example 6

### Decrease of Foreign Gene Transcripts with Heterologous Regulatory Elements

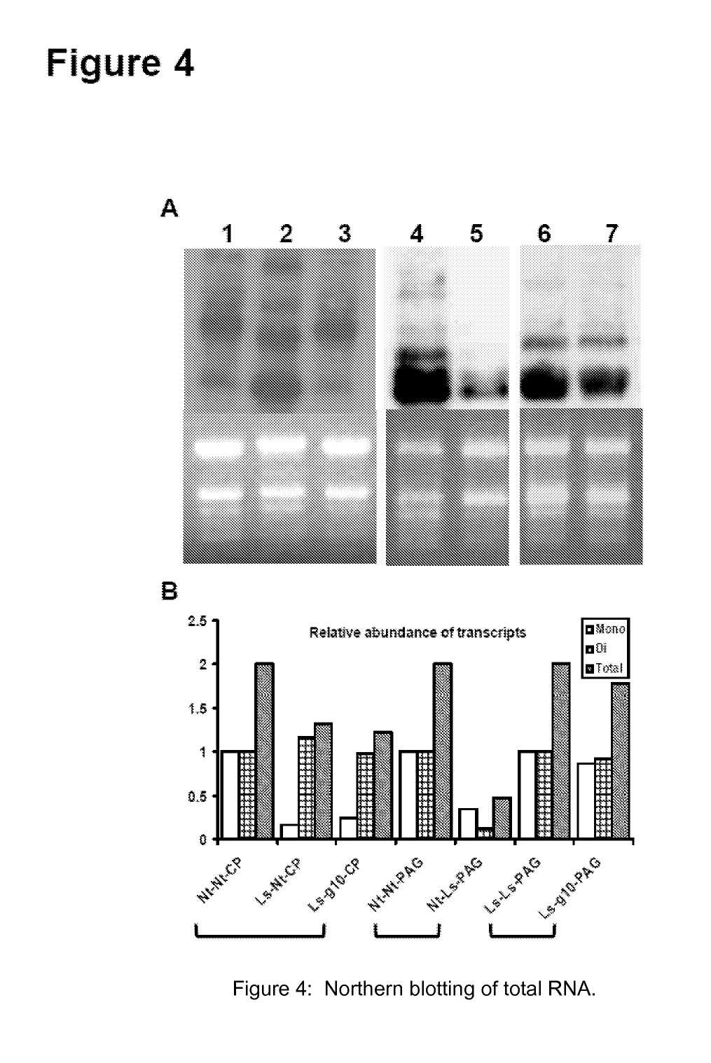

Total RNA was isolated from different transplastomic lines and northern blots were prepared to examine the transcript populations generated from various regulatory elements. Blots were probed with radiolabeled coding regions for CTB-Pins (FIG. 4A; lanes 1-3) and PA (lanes 4-7). Relative abundance of CTB-Pins and PA transcript species were assessed by densitometry (FIG. 4B). All of the plants under analysis carry two engineered promoters, one upstream of aadA and one upstream of the gene of interest, allowing for transcription of monocistrons, and also dicistrons arising from RNA polymerase read through of the upstream 3′ UTR element where present. While accumulation of dicistronic mRNA for CTB-Pins varied little, N. tabacum plants with endogenous psbA UTR expressed 84% more monocistronic mRNA and 66% total mRNA for CTB-Pins than L. sativa plants with the heterologous N. tabacum psbA UTR. As seen in transplastomic lines where genes are integrated within the ribosomal operon, a number of larger species were detectable using the CTB-Pins probe but were not quantified for this analysis. In PA lines where endogenous 5′ psbA was used, monocistronic transcript (FIG. 4, lanes 4 and 6) was elevated by 88% over those where heterologous psbA UTR was used as the regulatory element (FIG. 4, lane 5). Ethidium bromide staining of RNA gels is shown in the lower frame of FIG. 3A as an indicator of equal loading.

### Example 7

### Foreign Gene Transcripts with Endogenous 5′ UTR were Stabilized in Non-Polysomal Fractions

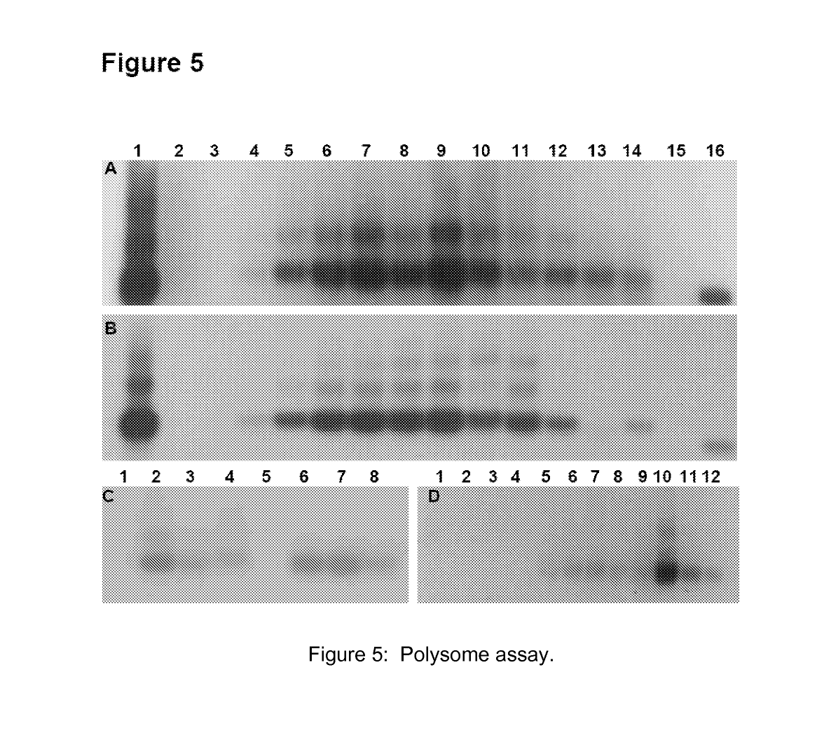

Total RNA was prepared from fractions separated through sucrose gradients to evaluate polysome association of foreign gene transcripts in L. sativa transformants that express CTB-Pins from the endogenous psbA UTR (Ls-Ls-CP) versus the N. tabacum psbA 5′ UTR (Ls-Nt-CP). The distribution of ribosomal RNAs in gradient fractions was similar between these two lines. Northern blots of 12 fractions collected from the bottom of the gradient were probed with the radiolabeled, full-length coding sequence for CTB-Pins to localize transplastomic transcripts. Blots prepared from all fractions off the gradient for Ls-Ls-CP or LS-Nt-CP lines show that the CTB-Pins transcript present in all fractions was predominantly monocistronic, although the dicistron is readily detectable and in some fractions abundant (FIGS. 5A & B). One larger CTB-Pins mRNA species was observed only in Ls-Nt-CP lines; this likely corresponds to a processed intermediate originating from the endogenous Prrn upstream of the insertion site. Foreign mRNA is more abundant in Ls-Ls-CP lines in the upper fractions suggesting that the transcript pool not associated with polysomes is stabilized in these lines (FIG. 5A). To account for variation in transfer and hybridization efficiency that can confound blot to blot comparisons, selected fractions were prepared from an independent isolation of these two lines and were blotted together to confirm this observation (FIG. 5C). Samples from untransformed plants and puromycin-treated controls are shown in FIG. 5D. Autoradiographs were used for densitometry. In Ls-Nt-CP line 22-37% of total signal was associated with the two non-polysomal fractions, whereas in the Ls-Ls-CP line 40-65% of the total signal was found in these fractions.

### Example 8

### Stromal RBPs Preferentially Associate with Endogenous psbA 5′ UTR Providing Transcript Stability

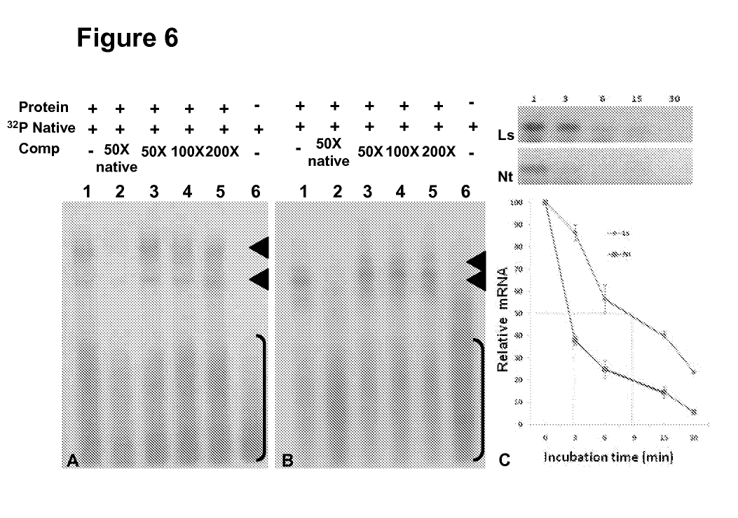

Extracts of plastid stromal proteins were prepared for L. sativa and N. tabacum as described in methods. Labeled, full length psbA 5′ UTRs specific to each system were transcribed in vitro as were the unlabeled competitors. Reactions were separated in native gels to evaluate the affinity of native UTRs for stromal protein binding in the presence of up to 200-fold molar excess of competitor species. As shown in FIG. 6, in the L. sativa (A) and N. tabacum (B) system, heterologous (unlabeled) psbA 5′ UTR was an ineffective competitor for association with RBPs in the stromal extracts. Two bands were observed in positive control reactions (lane 1) that were absent in reactions that did not contain stromal proteins (lane 6). The complexes migrated to the apparent molecular weight of 150 and 100 kDa in L. sativa extracts and 100 and 80 kDa in N. tabacum. The complexes formed between stromal proteins and labeled UTRs were disassociated by 50-fold molar excess of unlabeled endogenous UTR, but not by 50-, 100- or 200-fold excess of unlabeled heterologous psbA UTR. This result was consistent over 3 independent experiments in both backgrounds.

To determine if the RBP interaction affected mRNA turnover in these studies, we synthesized labeled LspsbA 5′ UTR CTB-Pins transcripts in vitro, incubated them in either L. sativa or N. tabacum stromal extracts and extracted total RNA over time. RNAs were electrophoresed, gels were dried and exposed to film for densitometric analysis. We found the half life of the radiolabeled transcript to be 8.4 min in the homologous

L. sativa extract while incubation of the transcript in the N. tabacum stromal extracts reduced the half life to 2.3 min, a 3.7 fold difference in the rate of turnover (FIG. 6C).

### Example 9

### CTB-Pins was Stable in All Transplastomic Lines Over Time

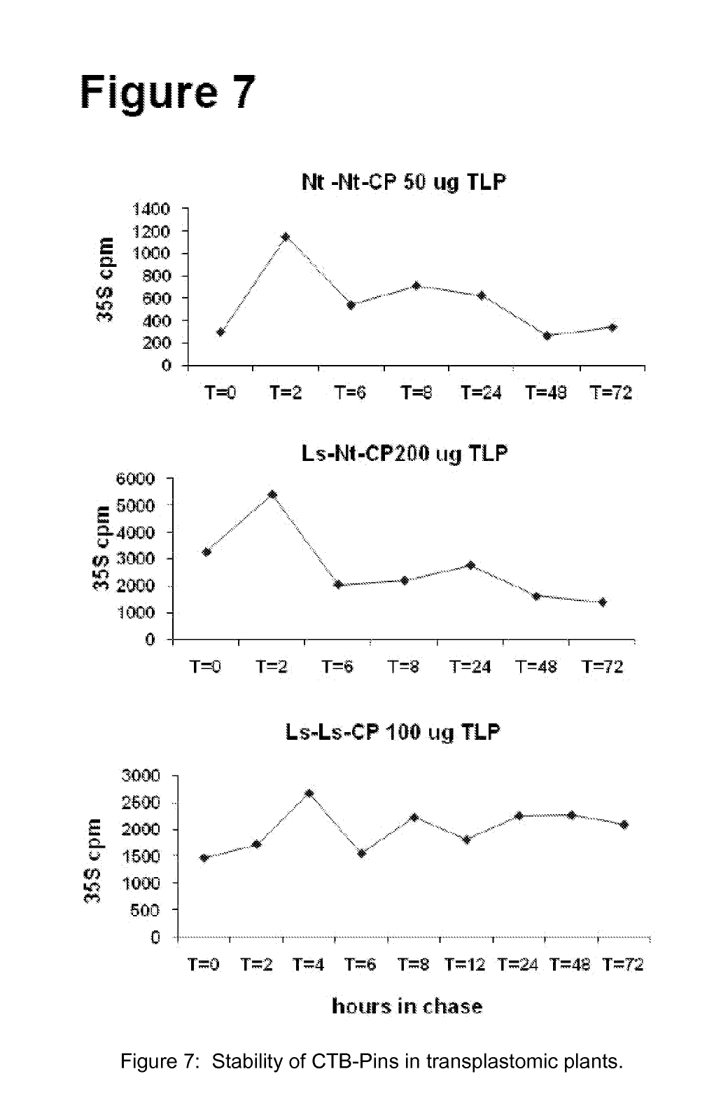

To determine if CTB-Pins protein was differentially stabilized in transplastomic lines we conducted protein labeling with ³⁵S and extracted leaf proteins at different time points throughout the chase period. Immunoprecipitation with CTB antibody was used to isolate the CTB-Pins protein away from the other labeled proteins translated during the pulse period. FIG. 7 shows result from ³⁵S immunoprecipitation of leaf samples. CPM (counts per minute) values for scintillation counting were low despite strong labeling detected in the total homogenates prior to immunoprecipitation of CTB. While there was some fluctuation in ³⁵S incorporation over time for all the transplastomic lines examined, stability of the foreign protein was unaffected by the use of heterologous regulatory elements.

### DISCUSSION of Examples 1-9

The foregoing examples disclose a rapid and reproducible method of chloroplast transformation in L. sativa. A plant system having direct shoot regeneration, species specific vector with endogenous regulatory elements, optimized DNA delivery, age of the explants and culture conditions were crucial in optimization of L. sativa chloroplast transformation. We found that L. sativa cv. Simpson elite had the highest direct-shoot regeneration potential, with maximum number of shoots (4.3), among the five cultivars examined in this study. The ability to produce direct shoot regeneration from somatic cells varies among the different cultivars of L. sativa (Xinrun and Connor, 1992). The induction of direct-shoot was highly dependent on the auxin and cytokinin ratio. Auxin or cytokinin alone did not induce shoot in any of the cultivars. Whereas leaf incubated on medium supplemented with 0.1 μg/ml NAA+0.2 μg/ml BAP induced maximum number of shoots in all cultivars, confirming that the optimum level of auxin-cytokinin balance is essential to induce maximum number of shoots in all the cultivars examined. Two types of organogenesis are known. In “direct organogenesis” the explants undergo a minimum proliferation before forming an organ such as shoot or root, whereas in “indirect organogenesis” explants undergo an extensive proliferation before it develops into shoot or root (Ovecka et al., 2000) and often requires re-determination of differentiated cells (dedifferentiation; George. et al., 2008). The L. sativa chloroplast transformation system reported here is established through direct organogenesis and therefore it is more rapid than the indirect organogenesis established through callus reported previously (Kanamoto et al., 2006; Lelivelt et al., 2005). Regeneration and efficiency of this system is comparable to tobacco, the most efficient transplastomic system developed so far. Such a rapid and reproducible transformation system has already facilitated expression of several vaccine antigens or autoantigens in lettuce chloroplasts.

A survey of the literature demonstrates that there is considerable interest in chloroplast transformation, both in the established system of N. tabacum and in crop species that have been more recalcitrant to transformation by this approach. More than 20 reports describe the generation of transplastomic crop plants through the use of species-specific flanking regions to facilitate homologous recombination between the shuttle vector and the plastid genome. However, the expression cassettes used in these experiments more contain heterologous regulatory elements either from another plant species, usually N. tabacum. In this study we have utilized untransformed L. sativa and N. tabacum plants along with a suite of transplastomic lines to investigate potential mechanisms that underlie the variation we have observed in foreign protein accumulation resulting from the use of endogenous or heterologous regulatory elements. The findings presented herein show that these differences may be influenced at multiple levels including transcription, mRNA processing, mRNA stability, and translation of foreign gene products.

Relative abundance of foreign gene transcripts in total RNA extractions of transplastomic lines were examined and the greatest variation in the monocistron pools in the different CTB-Pins lines were identified. Sequence alignments reveal that there is 100% identity between L. sativa and N. tabacum in the region of the ribosomal operon P1 promoter required for full PEP activity (−64 to +17; Suzuki et al., 2003). This is supported by similar levels of dicistron accumulation in the L. sativa and N. tabacum lines where transcription of this mRNA species is driven by foreign or native Prrn. Although this observation was consistent for Ls-Ls-PA and Ls-g10-PA lines, in Nt-Ls-PA, which carries the L. sativa Prrn, both the dicistron and monocistron were reduced. The core promoter elements shown to be sufficient for developmental regulation of psbA transcription are 90% identical between L. sativa and N. tabacum (−42 to +9; Hayashi et al., 2003) Therefore it seems unlikely that the differences that were observed in the foreign transcript pool result directly from an inability to achieve PEP-mediated synthesis due to divergence in promoter sequence and structure in the heterologous system, i.e. Ls-Nt-CP.

Based on sequence comparisons, the greatest variability upstream of the D1 coding region lies within the proposed stem-loop structure of the 5′ UTR that has been shown to be involved in transcript stability and translation efficiency in land plants (Alexander et al., 1998; Eibl et al., 1999; Shen et al., 2001; Zou et al., 2003). Over the entire sample set this region has a 61% sequence identity, and for L. sativa and N. tabacum the identity is 54.2%. The results provided herein from polysome analyses indicated that foreign transcripts in L. sativa lines which carried the endogenous 5′ UTR were more abundant in the upper fractions giving some insight into differences in foreign protein accumulation in the various lines used in this study.

It has been shown previously that the majority of psbA transcripts are not polysome associated in Hordeum vulgare, Spinacia oleracea and N. tabacum (Klein et al., 1988; Minami et al., 1988) and that this population is likely stabilized by RBPs in the stroma (Nakamura et al., 1999, 2001). A number of studies have investigated the cis-elements within the land plant psbA 5′ UTR to elucidate sequences which are required for the association of RBPs, and the role of these interactions in transcript stability, processing and initiation of translation on plastid ribosomes. Using synthetic psbA 5′ UTRs with specific site mutations and internal deletions it was determined that the stem loop region was a dispensable element as its exclusion did not affect translation of the fused lacZ reporter used in one study. An AU rich element (AU box) was identified between RBS1 and RBS2 that together were required and sufficient to initiate translation and proposed a role for the AU box as the primary target sequence for the binding of trans-acting factors (Hirose and Sugiura, 1996). Sequence analysis, combined with the results from RNA EMSA, suggest that the AU box and adjacent RBS sites are not responsible for differences observed in the experimental system. Between L. sativa and N. tabacum there was sequence identity of 95% over this region (20 bp), the variation generated by a single base change in RBS2. It was found, however, that this region has diverged in a subset of species, including representatives from each of the clades in our analysis, which have variable TA (AU) insertions at the 3′ end of the AU box (FIG. 1).

Subsequent analyses have suggested the presence of an endonucleolytic cleavage site that is protected upon the binding of protein factors. This site is localized to the predicted stem-loop region of the S. oleracea psbA 5′ UTR (−49/−48 relative to start of translation) and the binding interaction is sensitive to the secondary structure (Klaff et al., 1997; Alexander et al., 1998). Additional support for the role of the stem loop structure in stabilization of transcripts that include the psbA 5′ UTR is provided from transplastomic studies. Nicotiana tabacum plants were generated that express uidA (GUS) mRNA from chimeric genes where transcription was driven by Prrn with the full length endogenous psbA 5′ UTR (85 bases) or deletion variants upstream of the GUS coding region. In all experimental constructs mRNA abundance was reduced compared to the control despite transcription from identical promoters (Zou et al., 2003).

RNA EMSA assays using wild type stromal extracts demonstrated that the heterologous psbA 5′ UTR was not an effective competitor for binding factors that may be involved in transcript stability, and it was observed that the half life of the foreign transcript was reduced by up to 3.7 fold in the heterologous background. This strongly preferential binding of the endogenous UTR is attributed to the stem loop structural element as this is the primary region where significant sequence variation exists between L. sativa and N. tabacum. Conceivably in planta the ability of foreign gene transcripts equipped with heterologous UTR elements to compete for stabilization factors would be hampered by the presence of abundant endogenous psbA transcripts, leading to rapid turnover of foreign RNA species in the plastid.

The species-specific nature of protein factor binding to the psbA 5′ UTR has been further evaluated as a mechanism influencing foreign protein accumulation in transplastomic lines. We found that exchange of the full length UTR between L. sativa and N. tabacum resulted in a reduction in CTB-Pins and PA expression of at least 97% and 80%, respectively. This effect was consistent in young and mature leaves sampled at four time points. We did not detect a difference in the stability of CTB-Pins expressed from native or heterologous elements suggesting it was synthesis of the foreign protein, rather than degradation, which resulted in highly variable accumulation. CTB-Pins accumulated to much higher levels in older, but not senescent, leaves of Ls-Nt-CP plants but this could not compensate for the differences in overall expression as accumulation still showed a reduction of approximately 85% compared to N. tabacum plants with the endogenous UTR construct. PA lines did not accumulate foreign protein in older leaves to the same levels as CTB-Pins lines (FIG. 3). This is most likely due to differences in the solubility of the two proteins.

We estimate that CTB-Pins in fully expanded leaf tissue comprised 57% to 58% of the total leaf protein when harvested near the end of the light period and reached as high as 72%. The use of endogenous psbA 5′ UTR has led to the accumulation of numerous gene products in transplastomic N. tabacum (Verma et al., 2008), including proteins that had been previously unattainable at satisfactory levels using this technology (Fernandez-San Millan et al., 2003; Dhingra et al., 2004; Singh et al., 2008). We have now generated transplastomic L. sativa plants that accumulate abundant foreign protein. While the expression of first therapeutic protein produced in L. sativa was accomplished using the N. tabacum Prrn g10 system for expression of the gene of interest and the selectable marker (Ruhlman et al., 2007), the implementation of L. sativa specific regulatory elements has contributed to the development of a highly reproducible transformation system that generates transplastomic L. sativa plants expressing foreign proteins to high levels.

There are a great many factors to consider when designing transformation vectors for the generation transplastomic lines, particularly when the target is a new species for which there is no standardized approach. The use of species specific integration sequences (Verma and Daniell, 2007), codon optimization for plastid expression (Tregoning et al., 2003) and the inclusion of N-terminal stabilization sequences have been essential to the development of novel transplastomic lines (Ye et al., 2001; Leelavathi and Reddy, 2003). The emergent study of pentatricopeptide repeat (PPR) proteins in addition to well established research on RBPs have revealed that the interactions between protein factors and their cognate RNA sequences is highly specific. Our evaluation of UTR sequences from taxonomically diverse species for genes representing the various functional groups found in plastids combined with our experimental findings using the psbA 5′ UTR in particular argues for the use of species-specific regulatory elements for significant accumulation of foreign protein in transplastomic plants. This underscores the need for determining complete chloroplast genome sequences of crop species. It is equally important to optimize regeneration and tissue culture conditions by examining growth hormone requirements to achieve reproducible and rapid transformation of crop species.

### METHODS for Examples 1-9

Optimization of Direct Shoot Regeneration

Seeds of L. sativa cultivars (New England Seed Co., Hartford, Conn.) were disinfected in a solution of 30% commercial bleach with 0.01%, Tween-20 for 5 min, rinsed five times in sterile water and placed on half strength Murashige and Skoog (MS; Murashige and Skoog, 1962) medium solidified with 0.6% Phytablend® (Caisson, N. Logan, Utah). After 21 days, fully expanded first leaves were dissected into 0.8 cm ² and placed adaxial side on the medium. The culture medium was composed of MS basal salts, 3% sucrose and 0.6% Phytablend. Plant growth regulators were added to the medium as shown in table 1 and pH was adjusted to 5.8 prior autoclaving. Cultures were maintained at 26±2° C. at 40 μE m² s⁻¹ photon density; 16 hrs light/8 hrs dark.

Genomic Analyses

Sequences of IGS upstream from genes representing different functional groups were extracted from complete plastid genomes on GenBank for 20 species representing most major clades of angiosperms (see supplemental tables 1 & 2 for taxa and genes). Alignments were anchored by the inclusion of 100 bases from the coding region of adjacent genes. Sequences were aligned using MUSCLE (Edgar, 2004), followed by manual adjustment in Geneious (http://www.geneious.com/). Sequence identity was calculated for the region encompassed by 200 bases upstream of the translation start codon. For comparison sequence identity was also determined for the aligned coding regions. Geneious was used to conduct motif searches for functional domains such as promoter and cis translation elements and to calculate sequence identities for selected regions.

Vector Construction

The pUC-based L. sativa long flanking plasmid (pLS-LF) was used to integrate foreign genes into the intergenic spacer between tRNA-Ile and tRNA-Ala genes of the plastome IR. Details of this vector, the g10 CTB-Pins expression cassette and the pLD-CtV-5CP N. tabacum transformation vector are published (Ruhlman et al., 2007). The L. sativa native Prrn and 3′ rbcL for the expression of aadA from a GGAG RBS were amplified using total genomic L. sativa DNA with sequence specific primers and assembled in pBSSK+ vector. The aadA cassette was subcloned into pLS-LF at PvulI site and the resulting vector is named as pLsDV. The L. sativa native psbA 5′ and 3′ UTR were PCR amplified and cloned in pBSSK+vector to make an intermediate vector with multiple cloning site in between psbA 5′ and 3′ UTR resulting in pDVI-1. The CTB-Pins sequence and pag sequence was cloned at NdeI and XbaI site of pDVI-1 vector. The CTB-Pins and pag expression cassette with psbA 5′ and 3′ UTR was released by digestion with SalI and NotI and ligated into the pLsDV vector. A transformation cassette for the generation of transplastomic L. sativa plants that express CTB-Pins from N. tabacum psbA 5′ and 3′ UTR was assembled by digestion of the pLD-CtV-5CP plasmid with SalI and XbaI to release the CTB-Pins coding region plus the N. tabacum psbA 5′ UTR. This fragment was ligated into a pUC intermediate plasmid upstream of the N. tabacum psbA 3′ UTR. Nucleotide sequence of the intermediate plasmid pUC-NtUTR-CTB-Pins was confirmed. The cassette was released by digestion with SalI and SnaBI and ligated into the modified pLsDV vector digested by SalI and EcoRV. The pag coding sequence was released from pLD VK (Koya et al., 2005) plasmid by digesting with NdeI and NotI and cloned in pLsg10 CTB pins vector. All cloning steps were carried out in E. coli according to the methods of Sambrook and Russel (2001).

Bombardment and Selection of Transplastomic Lettuce

Seeds of L. sativa cv. Simpson elite (New England Seed Co.) were surface sterilized MS media solidified with 6 g L⁻¹ Phytablend® (Caisson). Young, fully expanded leaves ˜4 cm² were placed adaxial side up on antibiotic free L. sativa regeneration (LR) media (Kanamoto et al., 2006). Leaves were bombarded with 0.6 μm gold particles (BioRad) coated with one of the plastid transformation vectors shown in FIG. 2 and bombardments were carried out using the PDS-1000/He Biolistic® device employing 900 psi rupture disks and a target distance of 6 cm as described by Kumar and Daniell (2004). Bombarded leaf samples were held in dark at 25° C. for two days prior to explant of 0.5 cm² pieces, adaxial side down onto modified LR media containing 0.1 μg mL⁻¹ NAA and 0.2 682 g mL⁻¹ BAP with 50 μg mL⁻¹ spectinomycin dihydrochloride. Primary regenerants were screened by PCR for the transplastomic event and positive shoots were subjected to an additional regeneration cycle on the same selective media. Following the second regeneration shoots were rooted in half strength MS media 100 μg mL⁻¹ spectinomycin. Plants were propagated by rooting of nodal sections in half strength, hormone free MS with spectinomycin. Rooted cuttings were hardened in Jiffy® peat pots before transfer to the greenhouse for seed production. T1 seeds were harvested and plated on MS media with 100 μg mL⁻¹ spectinomycin along with wild type L. sativa to confirm maternal inheritance of plastid transgenes. Nicotiana tabacum plants included in analyses are reported in Ruhlman et al. (2007) and Koya et al. (2005). Transplastomic L. sativa plants expressing CTB-Pins from endogenous psbA 5′ and 3′ UTR were contributed by D. Burberry.

PCR Screening and Southern Blotting

Genomic DNA isolated from primary transformants was analyzed by PCR using primers 16SF (5′CAGCAGCCGCGGTAATACAGAGGA3′) and 3M (Singh et al., 2009). For Southern blotting, genomic DNA was isolated from young, in vitro grown leaves ground in liquid N₂ with chilled, sterile mortar and pestle, and extraction was carried out using a QIAGEN DNeasy® Plant mini kit (#69104) according to the manufacturer's protocol. Five micrograms of total DNA was digested to completion with BgIII (g10) or AfIIII (Nt-UTR) for CTB-Pins lines and SmaI for all PA lines, the resulting fragments were separated on 0.8% TAE-agarose gels and transferred to nylon membranes by capillary action. Plastid flanking sequence probe (1.3 kb) was amplified by PCR from L. sativa genomic DNA. PCR product was column purified and labeled probe was generated by incubation with α-³²P-dCTP and Ready-To-Go™ DNA Labeling Beads (GE Healthcare). Blots were pre-hybridized for one hour at 68° C. in QuikHyb® reagent (Stratagene, Cedar Creek, Tex.). Blots were hybridized for 1 hr 68° C., washed twice at 37° C. in 2×SSC (0.3 M sodium chloride, 30 mM sodium citrate, pH 7) and twice at 65° C. in 0.1×SSC. Radiolabeled blots were exposed to film on intensifying screens at −80° C. for 16 hrs.

Western Blot and Densitometric Analysis

Second generation (T1) CTB-Pins transplastomic L. sativa and N. tabacum were raised in the UCF greenhouse. Young, mature and older fully expanded leaves from ˜8 week old plants were harvested in August at 5 am, 10 am, 2 pm and 6 pm, ground in liquid N₂ and stored at −80° C. Approximately 100 mg of leaf tissue was suspended in five volumes of protein extraction buffer (100 mM NaCl, 10 mM EDTA, 200 mM Tris-HCl pH 8, 0.1% Triton X-100, 100 mM DTT, 400 mM sucrose, 2 mM PMSF). Extractions were vortexed vigorously for 20 min at 4° C. prior to determination of total protein using Bio-Rad Protein Assay Reagent. Total leaf proteins along with 100, 200, 400 and 600 ng of purified bacterial CTB. (Sigma, St. Louis, Mo.) were separated by sodium dodecylsulphate-polyacrylamide gel electrophoresis (SDS-PAGE) and transferred to nitrocellulose membranes for immunoblotting, according to Kumar and Daniell (2004). Immunoblotting with anti-CT primary antibody (1:3, 500, Sigma) and HRP-conjugated goat anti-rabbit secondary antibody (1:4,000, Southern Biotech, Birmingham, AL) was employed for densitometric analysis. A SuperSignal® West Pico HRP Substrate Kit (Pierce, Rockford, Ill.) was used for detection of chemiluminescence signal by exposure to film. Tissue collected at the latest developmental stage was prepared as above and subjected to SDS-PAGE along with CTB standards ranging from 0.5 to 3 μg. Gels were stained with Coomassie blue and used for densitometric quantitation.

Estimation of PA Protein Using ELISA

T1 PA transplastomic L. sativa and N. tabacum were raised in the UCF greenhouse. Young, mature and older, fully expanded leaves from ˜8 week old plants were harvested at 6 am, 12 pm, 6 pm and 3 am, ground in liquid N₂ and stored at −80° C. ELISA of leaf extract supernatant was performed in duplicate in a 96-Well EIA/RIA plate (Costar, Corning Inc. NY), along with purified PA as standard (courteously provided by Dr. Stephen H Leppla, NIH, Bethesda, Md.) The standard and samples were diluted in 15 mM Na₂CO₃, 35 mM NaHCO₃, 3 mM NaN₃, pH 9.6. PA standard ranging from 5 ng ml⁻¹ to 400 ng ml⁻¹ and sample dilutions of 1:10,000, 1:20,000 and 1:40,000 were loaded into the wells and incubated at 4° C. overnight. The plate was blocked with 3% fat-free milk in phosphate buffer saline and 0.05% Tween-20 (PBST), incubated at 37° C. for 1 hr and washed three times each with PBST and water. Primary monoclonal anti-PA (1:3,000) in PBST was loaded into wells and incubated at 37° C. for 1 hr. The wells were then washed with PBST and water as above. HRP-conjugated goat anti-rabbit (1:5, 000; Southern Biotech) was, incubated at 37° C. for 1 hr followed by washing with PBST and water. 100 μl of 3.3′, 5.5′-tetramethyl benzidine (TMB) substrate was loaded to each well and incubated at 24° C. for 10-15 min. The reaction was terminated by addition of 50 μl of 2N H₂SO₄. Plate was read at 450 nm using a plate reader (Model 680, Bio-Rad).

Plastid Isolation

Intact N. tabacum plastids were isolated according to Yukawa et al. (2007). Fully expanded green leaves (200 g) were collected from 4-5 week old greenhouse grown N. tabacum cv TN90. Leaves were homogenized in 50 g batches with 150 mL MCB1. Homogenates were filtered through 4 layers of cheesecloth then 2 layers of Miracloth (Calbiochem) and centrifuged for 5 min at 1,000×g in a Sorvall SS-34 fixed angle rotor at 4° C. Pellets were resuspended by gentle agitation with a soft paintbrush in 30 mL MCB1 and 5 mL was layered onto 25 mL Percoll (Sigma) gradients (20%-50%-80% in MCB1). Gradients were centrifuged for 10 min at 10,000×g in L-90K ultracentrifuge SW32-ti rotor; 2° C. The lower dark green band containing intact plastids was harvested, washed in three volumes MCB2. Plastids were collected by 1 min centrifugation at 600×g in Sorvall SS-34 at 4° C. Plastid pellet was resuspended in minimal volume of EMSA binding buffer (Alexander et al., 1998) with rigorous agitation to disrupt plastid envelopes. Membranes were sedimented by centrifugation in SS-34 rotor; 27,000×g for 15 min. Stromal extracts were adjusted to 15% glycerol, aliquoted and stored at −80° C.

Intact L. sativa plastids were isolated according to Gruissem et al. (1986). Fully expanded green leaves (200 g) of 6-8 week old hydroponically grown L. sativa cv longifolia were collected from the greenhouse and were homogenized in 50 g batches with 150 mL 1×GM buffer. Homogenates were filtered, centrifuged and pellets were resuspended as above in 1×GM buffer; 5 mL was layered onto 25 mL PCBF density gradients. Gradients were centrifuged for 20 min at 8,100×g. at 2° C. The lower dark green band containing intact plastids was harvested, washed in 2 volumes 1×GM buffer. Plastids were collected by 3 min centrifugation at 1,500×g at 4° C. Plastid pellet was resuspended in minimal volume of the above EMSA binding buffer and preserved as for N. tabacum

In vitro Transcription of Radiolabeled Transcripts

Plasmids (pBluescript SK+, Stratagene) containing the psbA 5′ UTR were digested with NdeI (L. sativa) or NcoI (N. tabacum) to generate linearized templates for T7 in vitro transcription using the MAXIscript® Kit (Ambion Inc, Austin Tex.) according to manufacturer's instructions. For mRNA turnover the L. sativa UTR-CTB-Pins plasmid was digested at the XbaI site situated at the 3′ end of CTB-Pins. For labeled UTR species uridine triphosphate (UTP) was replaced with 3.125 μM α-³²P UTP (Perkin Elmer, Waltham, Mass.). Reaction products were separated by denaturing polyacrylamide and eluted following manufacturer's instructions. Following ethanol precipitation supernatants were discarded and RNA pellets were vacuum dried. Pellets were resuspended 50 μL nuclease-free water and quantified by spectrophotometry and liquid scintillation counting. Single use aliquots were prepared and stored at −80° C.

Electrophoretic Mobility Shift and mRNA Turnover

Competitive RNA EMSA was adapted from Alexander et al. (1998). Stromal extracts were thawed on ice and total protein content was determined using the Bio-Rad Reagent (Bio-Rad). Stromal proteins of N. tabacum (20 μg) or L. sativa (40 μg) were incubated with 5 fmoles of endogenous radiolabeled psbA 5′UTR, with or without unlabeled competitor psbA 5′ UTR. All reactions were supplemented with 0.5 μg uL⁻¹ yeast tRNA (Ambion) to reduce non-specific binding and total volume was adjusted to 20 μL using EMSA binding assay buffer. Control reactions included no competition, competition with 50× molar excess of unlabeled native UTR and labeled probe only (no protein). Experimental reactions contained 50×, 100× and 200× molar excess of unlabeled non-native competitor UTR (i.e. N. tabacum protein with L. sativa UTR as competitor). Competitors were added 5 min prior to the labeled probe. Reactions were allowed to proceed for 15 min at 22° C. in the presence of labeled probe. 4 μL of 5× non-denaturing gel loading buffer was added (225 mM Tris-HCl, pH 6.8, 50% glycerol, 0.05% bromophenol blue) and reactions were separated through 8% polyacrylamide.

For degradation analysis of the labeled Ls-5′UTR-CTB-Pins transcript in stromal extracts the no competition control reaction described above was used. At the designated time points total RNA was isolated by phenol:chloroform as described below and extracts were separated by electrophoresis. Gels were vacuum dried and exposed to film for 6-12 hrs for analysis of binding reactions.

Polyribosome Association Assay and Northern Blotting

Polysome assay was adapted from Barkan et al., (1988). Green leaves from in vitro grown plants were ground to a fine powder in liquid N₂. Approximately 300 mg of each sample was transferred to a 2 mL tube and vortexed with 1 mL extraction buffer (0.2 M Tris HCl pH 9, 0.2 M KCl, 35 mM MgCl2, 25 mM EGTA pH 8.3, 1% Triton X100, 2% polyoxyethylene-10-tridecylether) with 0.5 mg mL⁻¹ heparin, 100 μg mL⁻¹ chloramphicol, 25 μg mL⁻¹ cyclohexamide. Homogenates were forced gently through glass wool packed in 3 mL syringe into microcentrifuge tube on ice and extracts were held on ice for 10 min. Extracts were centrifuged at 17,900×g for 5 min at 4° C. Supernatants were transferred to new tubes and 1/20^{th} volume of 10% sodium deoxycholate was added. Control reactions were incubated with puromycin (3 mg mL⁻¹) at 37° C. for 10 min. Reactions were held on ice for 5 min prior to centrifugation at 17 900×g for 15 min at 4° C. Supernatants (500 μL) were layered onto 15%-30%-40%-50% sucrose gradients in 10× salts (0.4 M Tris-HCl, pH 8, 0.2 M KCl, 0.1 M MgCl₂) prepared in Beckman ultraclear ½×2 inch tubes (13×51 mm; 344057). One aliquot of supernatant from each sample was reserved for isolation of total RNA. Gradients were centrifuged at 4° C. in SW55-ti rotor for 65 min at 192,000×g. Fractions of approximately 500 μL were collected into microfuge tubes containing 50 μL of 5% SDS and 0.2 M EDTA pH 8, by puncturing gradient tubes with an 18 gage needle. One volume of phenol:chloroform:isoamyl (25:24:1) was added to each fraction and vortexed then centrifuged at 17,900×g for 5 min. The aqueous phase was transferred to a new tube, two volumes absolute ethanol was added and mixed by inversion. RNA was pelleted by centrifugation, supernatant was discarded and pellets were dried under vacuum. Pellets were resuspended in 30 μL Tris-EDTA. RNA sample buffer (80 mM MOPS, pH 7, 4mM EDTA, 0.9 M formaldehyde, 20% glycerol, 30.1% formamide, 5 mM sodium acetate, 0.25% bromophenol blue) was added and fractions were separated in formaldehyde-agarose gels under denaturing conditions. Gels were washed in RNase-free water and RNA was transferred to nylon membranes (Nytran SPC, Whatman Inc., Sanford Me.) by capillary action in 20×SSC. Membranes were rinsed in RNase-free water and fixed by UV crosslinking. The full length CTB-Pins coding region was used to generate α-³²P-labeled, single stranded DNA probes according to the procedure described above for Southern blotting. Pre-hybridization and hybridization steps for polyribosome blots were carried out in Denhardt's buffer. Blots of total RNA extractions were hybridized in QuikHyb (Stratagene). For analysis of total transcripts, RNA was extracted using the Qiagen RNeasy®Mini Kit (Qiagen), and quantified by spectrophotometry. Total RNA, 2.5 μg, was prepared electrophoresed and blotted as described above.

Pulse-Chase Labeling and Immunopreciptation

Fully expanded leaves of L. sativa and N. tabacum transplastomic lines were cut into 5 mm² explants under half strength MS media with a sharp, sterile blade. Explants, approximately 50 per sample, were placed in a vacuum apparatus containing 10 mL MS media with 0.05% Tween 20 and 1 mCi EXPRESS ³⁵S protein labeling mix (PerkinElmer). Explants were infiltrated for 90 sec, transferred to a Petri dish along with the media and incubated in the light for 1 hr. Explants were removed from isotope media, rinsed with sterile water and transferred to the vacuum apparatus with fresh media without isotope containing 10 mM methionine. Infiltration was repeated and explants and media were transferred to a Petri dish for duration of chase period under 16 hrs light and 8 hrs dark in the growth chamber. At the time intervals shown in results, explants (5-6 each; ˜25 mg) were removed from the media, blotted on tissue paper and transferred to microcentrifuge tubes. Samples were frozen in liquid nitrogen and stored at −80° C. until the end of the chase period.

Samples were ground to a fine powder using a glass pestle with liquid N₂, in tubes. Approximately eight volumes of homogenization buffer (200 mM Tris-HCl pH 8, 100 mM NaCl, 10 mM EDTA, 0.1% SDS, 0.05% Tween 20, 2 mM PMSF) was added to each sample followed by vortexing with a micropestle. Total protein in homogenates was quantified using the Bio-Rad Reagent. An equal amount of total protein was taken from each sample and diluted to 1 mL in PBS. Primary antibody (a-cholera toxin from rabbit, 1:500, Sigma) was added to each sample and tubes were placed on a rocker at 4° C. for 4-6 hrs. To each tube 40 μL protein A-agarose (Santa Cruz Biotech, Santa Cruz, Calif.) was added and samples were incubated on the rocker at 4° C. for 16 hrs. Samples were pelleted by pulse centrifugation and aspirated. Pellets were washed three times with 500 μL PBS. Final pellet was suspended in 50 μL electrophoresis sample buffer (90 mM Tris HCl pH 6.8, 20% glycerol, 2% SDS, 0.02% Bromophenol blue, 100 mM dithiolthreitol) and boiled for 2 min. Samples were centrifuged and half of the supernatant was used for scintillation counting, while the other half was analyzed by SDS-PAGE. SDS gels were dried and exposed to film to visualize immunoprecipitated, labeled protein.

In reviewing the detailed disclosure which follows, and the specification more generally, it should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

TABLE 1
Regeneration efficiency in L. sativa cultivars

| | CULTIVAR | | | | | | | | | |
|---|---|---|---|---|---|---|---|---|---|---|
| | LE | | LG | | LL | | LR | | LS | |
| PGRs (μg ml⁻¹) | Mean SE | EFF | Mean SE | EFF | Mean SE | EFF | Mean SE | EFF | Mean ± SE | EFF |
| NAA 0.1 | | 0 | | 0 | | 0 | | 0 | | 0 |
| NAA 0.05 | 3.8 ± 0.9 | 0.63 | 3.8 ± 0.6 | 0.63 | 0.4 ± 0.12 | 0.06 | 7.2 ± 0.1 | 1.20 | 9.6 ± 0.1 | 1.60 |
| BAP 0.1 | | | | | | | | | | |
| NAA 0.05 | 4.4 ± 0.0 | 0.73 | 4.2 ± 0.2 | 0.70 | 1.0 ± 0.21 | 0.16 | 8.4 ± 0.2 | 1.40 | 11.2 ± 0.4 | 1.86 |
| BAP 0.2 | | | | | | | | | | |
| NAA 0.1 | 2.0 ± 0.1 | 0.33 | 5.0 ± 0.0 | 0.83 | 1.2 ± 0.2 | 0.20 | 6.0 ± 0.5 | 1.00 | 6.6 ± 0.1 | 1.10 |
| BAP 0.1 | | | | | | | | | | |
| NAA 0.1 | 7.2 ± 0.4 | 1.20 | 8.8 ± 0.3 | 1.46 | 2.6 ± 0.1 | 0.43 | 15.2 ± 0.0 | 2.53 | 25.8 ± 0.9 | 4.30 |
| BAP 0.2 | | | | | | | | | | |
| NAA 0.2 | 1.6 ± 0.1 | 0.26 | 1.4 ± 0.1 | 0.23 | | 0 | 3.8 ± 0.0 | 0.63 | 3.6 ± 0.0 | 0.60 |
| BAP 0.1 | | | | | | | | | | |
| NAA 0.2 | 1.2 ± 0.9 | 0.20 | 1.8 ± 0.2 | 0.30 | | 0 | 4.6 ± 0.4 | 0.76 | 4.8 ± 0.9 | 0.80 |
| BAP 0.2 | | | | | | | | | | |
| BAP 0.1 | | 0 | | 0 | | 0 | | 0 | | 0 |

EFF: Number of regenerated shoots/total explants

TABLE 2
RNA genes
Ribosomal RNA genes
rrn4.5*, rrn5*, rrn16*, rrn23*
Transfer RNA genes
trnA(UGC)*†, trnC(GCA), trnD(GUC), trnE(UUC), trnF(GGA),
trnG(GCC), trnH(GUG), trnI(CAU)*,trnI(GAU)*†, trnK(UUU)†,
trnL(CAA)*, trnL(UAA)†, trnL(UAC), trnfM(CAU), trnM(CAU),
trnN(GUU)*, trnP(UGG), trnQ(UUG), trnR(ACG)*, trnR(UCU),
trnS(GCU), trnS(GGA), trnS(UGA), trnT(GGU), trnT(UGU),
trnV(GAC)*, trnV(UAC)†, trnW(CCA), trnY(GUA)
Polypeptide genes
Ribosomal protein genes (larger subunit)
Rps2*†, rpl14, rpl16†, rpl20, rpl23*, rpl32, rpl33, rpl33, rpl36
Ribosomal protein genes (smaller subunit)
Rps2, rps3, rps4, rps7*, rps8, rps11, rps12*‡, rps14, rps15,
rps16†, rps18, rps19§
Transcription/translation apparatus genes: rpoA, rpoB, rpoC1†,
rpoC2, infA
Acetyl-CoA carboxylase: accD
ATP-dependent protease: clpP‡
ATP synthase: atpA, atpB, atpE, atpF†, atpH, atpI
Cytochrome b/f: petA, petB†, petD†, petG, petL, petN
Cytochrome c biogenesis: ccsA
Membrane protein: cemA
NADH dehydrogenase: ndhA†, ndhB*†, ndhC, ndhD, ndhE,
ndhF, ndhG, ndhH, ndhi, ndhJ, ndhK
Photosystem I: psaA, psaB, psaC, psaI,psaJ
Photosystem II: psbA, psbB, psbC, psbD, psbE, psbF, psbH,
psbI, psbJ, psbK, psbL, psbM, psbN, psbT, psbZ
Rubisco: rbcL
Maturase: matK [as introns in trnK(UUU)
Conserved reading frames: ycf 1, ycf2*, ycf 3‡, ycf 4
NADH, reduced nicotinamide adenine dinucleotide; Rubisco, ribulose-1,5-bisphosphate carboxylase/oxygenase.
*Gene duplicated in the inverted repeat.
†Gene with one introns.
‡Gene with two introns.
§Gene truncated in IRa.

TABLE 3

| | | Accession | Genome |
|---|---|---|---|
| Species | Common Name | Number | size (bp) |
| Agrostis stolonifera | Creeping bent grass | NC_008591 | 136,584 |
| Citrus sinensis | Orange | NC_008334 | 160,129 |
| Coffea arabica | Coffee | NC_008535 | 155,189 |
| Cucumis sativus | Cucumber | NC_007144 | 155,293 |
| | | DQ119058 | 155,527 |
| Daucus carota | Carrot | NC_008325 | 155,911 |
| Eucalyptus globulus | Eucalyptus | NC_008115 | 160,286 |
| Glycine max | Soybean | NC_007942 | 152,218 |
| Gossypium barbsfense | Sea Island cotton | NC_008641 | 160,316 |
| Gossypium hirsutum | Upland cotton | NC_007944 | 160,301 |
| Helianthus annuus | Sunflower | NC_007977 | 151,104 |
| Hordeum vulgare | Barley | NC_008590 | 136,462 |
| Lactuca sativa | Lettuce | NC_007578 | 152,765 |
| | | DQ383816 | 152,772 |
| Manihot esculenta | Cassava | EU117376 | 161,453 |
| Nicotiana tabacum | Tobacco | Z00044 | 155,939 |
| Oryza nivara | Indian wild rice | NC_005973 | 134,494 |
| Oryza sativa | Rice | X15901 | 134,525 |
| | | AY522329 | 134,496 |
| | | AY522331 | 134,551 |
| Panax ginseng | Ginseng | NC_006290 | 156,318 |
| Phaseolus vulgaris | Kidney bean | NC_009259 | 150,285 |
| Pinus koraiensis | Korean pine | NC_004677 | 117,190 |
| Pinus thunbergii | Japanese black pine | NC_001631 | 119,707 |
| Populus alba | White poplar | NC_008235 | 156,505 |
| Populus trichocarpa | Black cottonwood | NC_009143 | 157,033 |
| Saccharum hybrid | Sugarcane hybrid | NC_005878 | 141,182 |
| Saccharum officinarum | Sugarcane | NC_006084 | 141,182 |
| Solanum | Ornamental nightshade | NC_007943 | 155,371 |
| bulbocastanum | | | |
| Solanum lycopersicum | Tomato | NC_007898 | 155,461 |
| Solanum tuberosum | Potato | NC_008096 | 155,298 |
| | | DQ231562 | 155,312 |
| Sorghum bicolor | Sorghum | NC_008602 | 140,754 |
| Spinacia oleracea | Spinach | NC_002202 | 150,725 |
| Triticum aestivum | Bread wheat | NC_002762 | 134,545 |
| Vitis vinifera | Wine grape | NC_007957 | 160,928 |
| Zea mays | Maize | NC_001666 | 140,384 |

### REFERENCES

Alexander, C., Faber, N. and Klaff, P. (1998) Characterization of protein-binding to the spinach chloroplast psbA mRNA 5′ untranslated region. Nucleic Acids Res. 26: 2265-2273.

Allison, L. A., Simon, L. D. and Maliga P. (1996) Deletion of rpoB reveals a second distinct transcription system in plastids of higher plants. EMBO J. 15: 2802-2809.

Bally, J., Nadai, M., Vitel, M., Rolland, A., Dumain, R. and Dubald, M. (2009) Plant physiological adaptations to the massive foreign protein synthesis occurring in recombinant chloroplasts. Plant Physiol. PMID: 19458113.

Barkan, A. (1988). Proteins encoded by a complex chloroplast transcription unit are each translated from both monocistronic and polycistronic mRNAs. EMBO J. 7: 2637-2644.

Bock, R. (2007) Plastid biotechnology: prospects for herbicide and insect resistance, metabolic engineering and molecular farming. Curr. Opin. Biotechnol. 18: 100-106.

Daniell, H., Lee, S. B., Grevich, J., Saski, C., Quesada-Vargas, T., Guda, C., Tomkins, J., Jansen, R. K. (2006) Complete chloroplast genome sequences of Solanum bulbocastanum, Solanum lycopersicum and comparative analyses with other Solanaceae genomes. Theor. Appl. Genet. 112: 1503-1518.

Daniell, H., Ruiz, O. N. and Dhingra A. (2005) Chloroplast genetic engineering to improve agronomic traits. Methods Mol. Biol. 286: 111-138.

Davoodi-Semiromi, A., Samson, N. and Daniell, H. (2009) The green vaccine. A global strategy to combat infectious and autoimmune diseases. Human Vaccines 5: 1-6.

Dhingra, A., Portis, A. R., Jr. and Daniell, H. (2004) Enhanced translation of a chloroplast-expressed rbcS gene restores small subunit levels and photosynthesis in nuclear rbcS antisense plants. Proc. Natl. Acad. Sci.USA 101: 6315-6320.

Edgar, R. C. (2004), MUSCLE: multiple sequence alignment with highaccuracy and high throughput. Nucleic Acids Res. 32: 1792-97.

Eibl, C., Zou, Z., Beck, A., Kim, M., Mullet, J. and Koop H. U. (1999) In vivo analysis of plastid psbA, rbcL and rp132 UTR elements by chloroplast transformation: Tobacco plastid gene expression is controlled by modulation of transcript levels and translation efficiency. Plant J. 19: 333-345.

Fernandez-San Millan, A., Mingo-Castel, A., Miller, M. and Daniell, H. (2003) A chloroplast transgenic approach to hyper-express and purify Human Serum Albumin, a protein highly susceptible to proteolytic degradation. Plant Biotechnol. J. 1:71-79.

George, E. F., Hall, M. A. and Klerk, G- J. K. (eds) (2008) Plant Propagation by Tissue Culture. Spinger, Basingstoke, UK.

Gruissem, W., Greenberg, G., Zurawski, G. and Hallick, R. B. (1986) Chloroplast gene expression and promoter identification in chloroplast extracts. Methods Enzymol. 118: 253-270.

Guda, C., Lee, S. B. and Daniell, H. (2000) Stable expression of a biodegradable protein-based polymer in tobacco chloroplasts. Plant Cell Rep. 19: 257-265.

Hayashi, K., Shiina, T., Ishii, N., Iwai, K., Ishizaki, Y., Morikawa, K. and Toyoshima, Y. (2003) A role of the -35 element in the initiation of transcription at psbA promoter in tobacco plastids. Plant Cell Physiol. 44: 334-341.

Hess, W. R. and Borner, T. (1999) Organellar RNA polymerases of higher plants. Int. Rev. Cytol. 190: 1-59.

Hirose, T, and Sugiura, M. (1996) Cis-acting elements and trans-acting factors for accurate translation of chloroplast psbA mRNAs: development of an in vitro translation system from tobacco chloroplasts. EMBO J. 15: 1687-1695.

Kanamoto, H., Yamashita, A., Asao, H., Okumura, S., Takase, H., Hattori, M., Yokota, A. and Tomizawa, K. (2006) Efficient and stable transformation of Lactuca sativa L. cv. Cisco (lettuce) plastids. Transgenic Res. 15: 205-217.

Klaff, P., Mundt, S. M. and Steger, G. (1997) Complex formation of the spinach chloroplast psbA mRNA 5′ untranslated region with proteins is dependent on the RNA structure. RNA 3: 1468-1479.

Klein, R. R., Mason, H. S. and Mullet, J. E. (1988) Light-regulated translation of chloroplast proteins. I. Transcripts of psaA-psaB, psbA, and rbcL are associated with polysomes in dark-grown and illuminated barley seedlings. J. Cell Biol. 106: 289-301.

Koya, V., Moayeri, M., Leppla, S. H. and Daniell, H. (2005) Plant-based vaccine: Mice immunized with chloroplast-derived anthrax Protective antigen survive anthrax lethal toxin challenge. Infect. Immun. 73: 8266-8274.

Kumar, S. and Daniell, H. (2004) Engineering the chloroplast genome for hyperexpression of human therapeutic proteins and vaccine antigens. Methods Mol. Biol. 267: 365-383.

Kuroda, H. and Maliga, P. (2001) Complementarity of the 16S rRNA penultimate stem with sequences downstream of the AUG destabilizes the plastid mRNAs. Nucleic Acids Res. 29: 970-975.

Leelavathi, S. and Reddy, V. S. (2003) Chloroplast expression of His-tagged GUS-fusions: a general strategy to overproduce and purify foreign proteins using transplastomic plants as bioreactors. Mol. Breed. 11: 49-58.

Lelivelt, C. L., McCabe, M. S., Newell, C. A., deSnoo, C. B., van Dun, K. M., Birch-Machin, I., Gray, J. C., Mills, K. H. and Nugent, J. M. (2005) Stable plastid transformation in lettuce (Lactuca sativa L.). Plant Mol. Biol. 58: 763-774.

Marder, J., Chapman, D., Telfer, A., Nixon, P. and Barber, J. (1987) Identification of psbA and psbD gene products, D1 and D2, as reaction centre proteins of photosystem 2. Plant Mol. Biol. 9: 325-333.

Meierhoff, K., Felder, S., Nakamura, T., Bechtold, N. and Schuster, G. (2003) HCF152, an Arabidopsis RNA binding pentatricopeptide repeat protein involved in the processing of chloroplast psbB-psbT-psbH-petB-petD RNAs. Plant Cell 15: 1480-1495.

Merhige, P. M., Both-Kim, D., Robida, M. D. and Hollingsworth, M. J. (2005) RNA-protein complexes that form in the spinach chloroplast atpl 5′ untranslated region can be divided into two subcomplexes, each comprised of unique cis-elements and trans-factors. Curr. Genet. 48: 256-264.

Minami, E., Shinohara, K., Kawakami, N., and Watanabe, A. (1988) Localization and properties of transcripts of psbA and rbcL genes in the stroma of spinach chloroplast. Plant Cell Physiol. 29: 1303-1309.

Murashige, T. and Skoog, A. (1962) A revised medium for rapid growth and bioassay with tobacco tissue cultures: Physiol. Plant. 15: 473-497.

Nakamura, T., Ohta, M., Sugiura, M. and Sugita, M. (1999) Chloroplast ribonucleoproteins are associated with both mRNAs and intron-containing precursor tRNAs. FEBS Lett. 460: 437-431.

Nakamura, T., Ohta, M., Sugiura, M. and Sugita M (2001) Chloroplast ribonucleoproteins function as a stabilizing factor of ribosome-free mRNAs in the stroma. J. Biol. Chem. 276: 147-152.

Nickelsen, J. (2003) Chloroplast RNA-binding proteins. Curr. Genet. 43: 392-399. Ovecka, M., Bobak, M. and Samaj, J. (2000) A comparative structural analysis of direct and indirect shoot regeneration of Papaver somniferum L. in vitro. J. Plant Physiol. 157: 281-289

Ruhlman, T., Ahangari, R., Devine, A., Samsam, M. and Daniell, H. (2007)

Expression of cholera toxin B-proinsulin fusion protein in lettuce and tobacco chloroplasts--oral administration protects against development of insulitis in non-obese diabetic mice. Plant Biotechnol. J. 5: 495-510.

Saski, C., Lee, S. B., Fjellheim, S., Guda, C., Jansen, R. K., Luo, H., Tomkins, J., Rognli, O. A., Daniell, H. and Clarke, J. L. (2007) Complete chloroplast genome sequences of Hordeum vulgare, Sorghum bicolor and Agrostis stolonifera, and comparative analyses with other grass genomes. Theor. Appl. Genet. 115: 571-590.

Schmitz-Linneweber, C. and Barkan, A. (2007) RNA splicing and RNA editing in chloroplasts. In Cell and Molecular Biology of Plastids. Ed. R. Bock. Springer, Berlin. pp 213-248.

Schmitz-Linneweber, C., Williams-Carrier, R. and Barkan, A. (2005) RNA immunoprecipitation and microarray analysis show a chloroplast pentatricopeptide repeat protein to be associated with the 5′ region of mRNAs whose translation it activates. Plant Cell 17: 2791-2804.

Schmitz-Linneweber, C., Williams-Carrier, R. E., Williams-Voelker, P. M., Kroeger T. S., Vichas, A. and Barkan, A. (2006) A pentatricopeptide repeat protein facilitates the trans-splicing of the maize chloroplast rps12 pre-mRNA. Plant Cell 18: 2650-2663.

Shen, Y., Danon, A. and Christopher, D. A. (2001) RNA binding-proteins interact specifically with the Arabidopsis chloroplast psbA mRNA 5′ untranslated region in

Singh, N. D., Ding, Y. and Daniell, H. (2009) Chloroplast-derived vaccine antigens and biopharmaceuticals: protocols for expression, purification, or oral delivery and functional evaluation. Methods Mol. Biol. 483: 163-192.

Singh, N. D., Li, M., Lee, S. B., Schnell, D. and Daniell, H. (2008) Arabidopsis Tic40 expression in tobacco chloroplasts results in massive proliferation of the inner envelope membrane and upregulation of associated proteins. Plant Cell 20: 3405-3417.

Staub, J. M., Garcia, B., Graves, J., Hajdukiewicz, P. T. J., Hunter, P., Nehra, N., Paradkar, V., Schlittler, M., Carroll, J. A., Spatola, Ward, D., Ye, G. and Russell, D. A. (2003) High-yield production of a human therapeutic protein in tobacco chloroplasts. Nat. Biotechnol. 19: 333-338.

Suzuki, J. Y., Sriraman, P., Svab, Z. and Maliga P (2003) Unique architecture of the plastid ribosomal RNA operon promoter recognized by the multisubunit RNA polymerase in tobacco and other higher plants. Plant Cell 15: 195-205.

Timme, R. E., Kuehl, J. V., Boore, J. L. and Jansen, R. K. (2007) A comparative analysis of the Lactuca and Helianthus (Asteraceae) plastid genomes: identification of divergent regions and categorization of shared repeats. Am. J. Bot. 94: 302-312.

Tregoning, J. S., Nixon, P., Kuroda, H., Svab, Z., Clare, S., Bowe, F., Fairweather, N., Ytterberg, J., van Wijk, K. J., Dougan, G. and Maliga, P. (2003) Expression of tetanus toxin Fragement C in tobacco chloroplasts. Nucleic Acids Res. 31: 1174-1179.

Verma, D., Samson, N. P., Koya, V. and Daniell, H. (2008) A protocol for expression of foreign genes in chloroplasts. Nat. Protoc. 3: 739-758.

Verma, D. and Daniell, H. (2007) Chloroplast vector systems for biotechnology applications. Plant Physiol. 145: 1129-1143.

Xinrun, Z. and Conner. A. J. (1992) Genotype effects on tissue culture response of lettuce cotyledons. J. Genet. Breed. 46: 287-290.

Yamamoto, Y. (2001) Quality Control of Photosystem II. Plant Cell Physiol. 42: 121-128.

Yang, J., Usack, L., Monde, R. A. and Stern, D. B. (1995) The 41 kDa protein component of the spinach chloroplast petD mRNA 3′ stem-loop:protein complex is a nuclear encoded chloroplast RNA-binding protein. Nucleic Acids Symp. Ser. 33: 237-239.

Ye, G. N, Hajdukiewicz, P. T. J.,Broyles, D., Rodriguez, D., Xu, C. W., Nehra, N. and Staub, J. M. (2001) Plastid-expressed 5-enolpyruvylshikimate-3-phosphate synthase genes provide high level glyphosate tolerance in tobacco. Plant J. 25: 261-270

Yukawa, M., Kuroda, H. and Sugiura, M. (2007) A new in vitro translation system for non-radioactive assay from tobacco chloroplasts: effect of pre-mRNA processing on translation in vitro. Plant J. 49: 337-376.

Zou, Z., Eibl, C., and Koop, H. U. (2003) The stem-loop region of the tobacco psbA 5′UTR is an important determinant of mRNA stability and translation efficiency. Mol. Genet. Genomics 269: 340-349.

The teachings of all references cited herein are incorporated herein in their entirety to the extent they are not inconsistent with the teachings herein. U.S. Patent Publication 20090022705 is specifically incorporated herein by reference, which provides discussion on edible plants and molecular plant transformation techniques and sequence identity calculations.

What is claimed is:

1. A stable plastid transformation and expression vector which comprises an expression cassette optimized for expression in a target plant species, the expression cassette comprising a regulatory sequence that is endogenous to said target plant species, a heterologous polynucleotide sequence of interest, transcription termination sequence functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome, and wherein the expression cassette optionally includes a heterologous marker sequence.

2. The vector of claim 1, wherein said regulatory sequence comprises at least a portion of a promoter sequence for plastid gene of said target plant species.

3. The vector of claim 1, wherein the target plant species is not tobacco.

4. The vector of claim 1, wherein said target plant species is lettuce.

5. The vector of claim 1, wherein said target plant species is a cereal crop, melon, legume, an oil crop, a tuber crop, a vegetable crop; a fruit crop, a fiber crop, an ornamental plant, a grass or tree.

6. The vector of claim 1, wherein the target plant species is maize, rice, soybean, wheat or cotton.

7. The vector of claim 1, wherein the target plant species excludes tobacco or rice.

8. The vector of claim 1, wherein said regulatory sequence, is a psbA 5′ UTR sequence.

9. The vector of claim 1, wherein said psbA 5′ UTR sequence is endogenous to L. sativa, S. lycopersicum, S. tuberosum, H. annuus, G. abyssinica, O. sativa, H. vulgare or Z. mays.

10. The vector of claim 1, wherein said regulatory sequence is a 5′ UTR sequence of a chloroplast gene endogenous to said target plant species.

11. The vector of claim 10, wherein said 5′ UTR sequence comprises a stem and loop sequence.

12. The vector of claim 11, wherein said stem and loop sequence is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

13. The vector of claim 10, wherein the 5′ UTR sequence comprises a ribosome binding site.

14. A plant transformed with the vector of claim 1.

15. A stable plastid transformation and expression vector for transformation of a target plastid genome, said vector comprises an expression cassette comprising, as operably linked components in the 5′ to the 3′ direction, a promoter operative in said plastid, a selectable marker sequence, an endogenous 5′ UTR sequence, a gene of interest, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

16. The vector of claim 15, wherein said endogenous 5′ UTR sequence is a Lactuca sativa psbA 5′ UTR sequence.

17. The vector of claim 15, wherein said endogenous 5′ UTR sequence is a Z. mays psbA 5′ UTR sequence.

18. The vector of claim 15, wherein said endogenous 5′ UTR sequence is an O. sativa psbA 5′ UTR sequence.

19. The vector of claim 15, wherein said endogenous 5′ UTR sequence is a T aestivum psbA 5′ UTR sequence.

20. The vector of claim 15, wherein said endogenous 5′ UTR sequence is a G. max psbA 5′ UTR sequence.

21. A transplastomic plant other than tobacco that comprises a plastid transformed to express a heterologous gene of interest regulated by a 5′ UTR sequence operably associated with said gene of interest, wherein said 5′ UTR is endogenous to said plant.

22. The plant of claim 21, wherein said heterologous gene of interest and 5′ UTR sequence are located on the same DNA strand with the 5′ UTR arranged in a 5′ direction relative to said heterologous gene of interest.
